(19) United States
(12) Patent Application Publication
(10) Pub. No.: US 2010/0113952 A1
(43) Pub. Date: May 6, 2010

(54) Apparatus and method for the identification of fake fingerprints

(76) Inventors: Daniel H. Raguin, Acton, MA (US); John F. Carver, Palm City, FL (US); Joe F. Arnold, Palm Beach Gardens, FL (US)

Correspondence Address: Kenneth J. LuKacher;South Winton Court, Suite 301, 3136 Winton Road South, Rochester, NY, 14623, US

(21) Appl. No.: 12/590,153

(22) Filed: Nov. 3, 2009

Related U.S. Application Data
(60) Provisional application No. 61/198,234, filed on Nov. 3, 2008.

(51) Int. Cl. A61B 5/0408; G01R 27/26; G01R 27/08; A61B 5/01

(52) U.S. Cl. 600/509; 324/663; 324/692; 600/549

(57) ABSTRACT

An apparatus for identifying fake fingerprints has electrodes disposed along a platen surface of a fingerprint scanner and whether or not the skin of one or more fingers presented to the surface are real and alive is determined in accordance with analysis of electrical signals received from the electrodes. Electronics of the apparatus determines one or more liveness parameter(s) in accordance with signals received from electrodes. Information from an image of the fingerprint may be used to select which electrode signals to use for liveness detection. To further confirm the presence of a live finger(s), additional liveness parameter(s) of the pulse and/or temperature may also be sensed. The skin may be one of in contact with the platen of the fingerprint scanner, separated from direct contact with the platen's electrodes by an insulating layer or a pad, or not in physical contact with the platen or a pad thereupon.

This application claims priority to U.S. Provisional Application No. 61/198,234, filed Nov. 3, 2008, which is herein incorporated by reference.

### FIELD OF THE INVENTION

The present invention relates to an apparatus (and method) for identifying fake fingerprints in a fingerprint scanner which images a single fingerprint, multiple fingerprints, or a large area of skin, and particularly to an apparatus having electrodes disposed along a platen surface for determining whether or not the finger skin presented to the platen surface for imaging is real and alive in accordance with analysis of electrical signals received from the electrodes. The signals detected by the electrodes may be used to determine the electrical impedance of the skin, for the detection of voltages associated with the heart beat of a human, for detection of conductivity changes, and/or to determine the temperature of the skin in relationship to the ambient temperature. To determine electrical impedance, the electrodes operate as antennas for sending signals at particular frequencies or ranges, and receiving signals having amplitude and phase to determine impedance. Electrodes operate with or without direct contact of skin with the apparatus. In the case of skin contact, the electrodes may be incorporated into an image enhancing material that may or may not be removable from the apparatus' platen. Further by incorporating the apparatus in a fingerprint scanner (or imaging system), either optical or ultrasonic, an image of fingerprint(s) is obtainable to determine where the finger print(s) are relative to the electrodes for use in determining which electrode signals are utilized for liveness detection. The identification of fake fingerprints of the present invention may be incorporated in conventional fingerprint scanners with the improvement being provided by the present invention's layout, pattern, or array of electrodes along the platen of the scanner for facilitating liveness detection when one or more multiple fingers are scanned.

### BACKGROUND OF THE INVENTION

Growing concerns regarding security have created a critical need to positively identify individuals through means that is tied to the actual individual and is not based or at least not solely based upon the individual holding forms of identification such as credit cards, driver's licenses, passports or other forms of printed identification. As such, biometric identification technologies such as fingerprint have become increasingly popular means for individual identification due to their ability to discriminate one person among billions since the fingerprints of even monozygotic (identical) twins differ sufficiently for positive discrimination. Despite its capability for low false-accept-rates (FAR) and false-reject-rates (FRR), biometric security systems, as with any security system, will present an incentive among certain individuals to spoof the system to gain access to something desired or to avoid being recognized.

The need to detect fake fingerprints (or in other words spoof attempts) has been recognized for many years. For example, fingerprint scanners that check the liveness of a subject's fingers have been proposed based on spectral analysis of the skin (e.g., U.S. Pat. Nos. 7,147,153 and 7,347,365), optical pulse detection (e.g., photoplethysmography as described by U.S. Pat. No. 6,483,929), and by detecting the blanching effect of skin induced by pressure (U.S. Pat. No. 4,728,186).

Liveness detection based upon electrical signal sensing has been proposed for use in fingerprints. U.S. Pat. Nos. 5,828,773 and 5,953,441 describe measuring the complex impedance Z at a fixed electrical frequency of the skin of a fingerprint using an array of miniature electrodes. These patents describe that a 256×256 array of electrodes across a 0.5″×0.5″ area (i.e., the electrodes have a 50.4 μm center-to-center spacing) can be used to create an electrical image of the fingerprint and that these electrodes may be coated with a 2 to 100 μm thick anisotropic dielectric coating that focuses the electrical fields and provides mechanical protection from finger contact and chemical resistance protection from oils of the finger and solvents used in the periodic cleaning of the finger platen. Further these patents describe that an indication of the liveness of the fingerprint may be obtained based upon the phase angle of the complex impedance measured. Such patents do not describe use of transparent electrodes in an optical fingerprint sensor, and limit their analysis of skin electrical signals to a fixed electrical frequency, and lithographic technology for fabricating the electrodes that is not scalable to large platen sizes capable of capturing surface topology images from two fingers, four fingers, or entire palm or hand.

U.S. Published Patent Application No. 2005/0281441 describes the use of sets of four electrodes in order to measure the complex impedance of the dermal skin which is minimally affected by the moisture content of the outer epidermal layer of the skin. This concept of using four electrodes for bioimpedance is also described by Hinton and Sayers of Solartron Analytical (Farnborough, Hampshire UK) in their white paper “Advanced Instruments for Bioimpedance Measurements,” dated 1998. Although U.S. Published Patent Application No. 2005/0281441 describes the benefit of analyzing the impedance of the skin at a range of electrical frequencies in order to check liveness, electrodes are neither integrated with an optical or ultrasound fingerprint scanner, nor associated with the layout of electrodes for the capture of signals from skin larger than the size of a fingertip.

U.S. Pat. No. 6,327,376 describes transparent electrodes in a single fingerprint scanner having an array of insulated transparent electrodes (for example composed of indium tin oxide—ITO) with a pitch of approximately 50 to 100 μm to capture the fingerprint image of a portion of skin based upon capacitance measurements. This patent also describes an optical system that can be incorporated into the apparatus for the purposes of liveness detection (e.g., measurement of pulse rate, or blood oxygen content). Although U.S. Pat. No. 6,327,376 describes use of electrodes for the capture of a fingerprint image and the use of an optical device for liveness detection, it does not describe use of electrodes for liveness detection.

U.S. Published Patent Application Nos. 2006/0159314 and 2006/0140456 describe the use of transparent electrodes integrated into a single-finger optical fingerprint scanner, where the electrodes are used to measure electrical characteristics of the fingerprint at substantially the same time as an optical image of the fingerprint is captured. The complex impedance Z of the finger is measured at a fixed electrical frequency between one or more electrode pairs and based upon the optical image of the fingerprint, the area of the fingerprint contacting a particular electrode pair is used to relate the impedance measured to a range of values characteristic of a human skin response. In the calculation of area, these Published Application Nos. 2006/0159314 and 2006/0140456 do not take into account the topology of the fingerprint and instead only consider the overall shadow region created by the fingerprint. Further, they only consider the case of electrode arrangements for a single-finger scanner, and electrodes and electronics that require the platen to be fixed (e.g., non-removable) as well as require the skin to be in direct contact with the electrodes. Further, these Published Application Nos. 2006/0159314 and 2006/0140456 consider only a fixed electrical frequency for measuring complex impedance Z of the skin and do not attempt to measure heartbeat or temperature of the skin. Transparent electrodes, e.g., ITO (indium tin oxide), for liveness detection in an apparatus that captures optical images of fingerprints are also discussed by U.S. Pat. No. 6,175,641, where such electrodes may or may not be overcoated by an insulating layer.

Although the prior art discussed above describes the use of electrodes for impedance-based liveness detection by incorporation of transparent electrodes in a single-finger optical fingerprint scanner, such prior art does not address the issues associated with the capture of multiple fingers or similar large areas of skin and/or any arrangement of electrodes to enable low-cost production of electrodes in platens. Furthermore, the prior art cited earlier neither addresses the use of material or a pad to improve optical or ultrasonic imaging of a fingerprint while enabling electrodes in the fingerprint scanner to perform a liveness check, nor the use of the image of the topology of the fingerprint in liveness detection, nor how the same electrodes can be used to check for a pulse as well as temperature of the skin and ambient temperature. Lastly, the prior art discussed herein does not address how liveness detection based upon electrical signals can be performed when the finger(s) or other portion of the skin is substantially separated from the fingerprint scanner (i.e., a touchless system).

### SUMMARY OF THE INVENTION

It is a feature of the present invention to provide improved identification of fake fingerprints in a multiple fingerprint scanner using electrodes for liveness detection, especially in layouts or arrangements which enable low-cost production of these electrodes, and which, if needed, may utilize the image of the fingerprints to identify the electrodes relative to imaged fingerprints for liveness detection.

It is another feature of the present invention to provide improved identification of fake fingerprints for a scanner for single or multiple fingerprints when the finger(s) or other portion of the skin is substantially separated from the fingerprint scanner's platen.

A further feature of the present invention is to provide improved identification of fake fingerprints in a scanner for imaging fingerprint(s) having a platen with electrodes for liveness detection which enhance optical or ultrasonic imaging of the fingerprint while enabling electrodes in the fingerprint scanner to perform a liveness check.

A still further feature of the present invention is to provide improved identification of fake fingerprints using electrode layouts or arrangement that require proper spatial orientation of fingers with respect to electrodes, or do not require spatial orientation of finger(s) with respect to electrodes, while maintaining low-cost production of these electrodes rather than locating a large number of finely spaced, expensive to fabricate, electrodes over the entire platen imaging area to enable liveness detection.

Briefly described, the present invention embodies a finger-print scanner (or scanning apparatus) having an imaging system for capturing an image of one or multiple fingerprints of one or more fingers via a platen of the scanner, in which a plurality of electrodes are disposed in or upon the platen, and electronics for detecting liveness of such one or more fingers in accordance with electrical signals provided from the electrodes. In one configuration where the image is of multiple fingerprints, the electrodes are disposed in a configuration in which different groups of electrodes are disposed for different ones of the fingers and the fingers are spatially oriented on the platen with respect to particular ones of the groups of the electrodes. In another configuration where the image is of multiple fingerprints, the electrodes along the platen are disposed in an array configuration and one or more of such electrodes is oriented on the platen to receive a signal from multiple ones of such fingers, and no particular spatial orientation of each of the fingers with respect to electrodes is needed.

The electrodes when upon the platen, rather than integrated in the platen, are either formed on the surface of the platen, or disposed in a pad situated upon (or adhesively attached to) the surface of the platen, in which the finger(s) of the subject are presented to the platen via contact with the pad. A member may be provided adjacent the platen or the pad having electrical leads which electrically connect by contacting electrical leads extending from each electrode, such that the leads along the member enable signals from electrodes (via their respective leads) to be communicated to the processor(s). This member is the case of the electrodes being provided in a pad upon the platen, may be a clamp member which retains the pad in place. The member is the case of the electrodes being provided in or on the platen may be a cover member of a housing of the scanner. The leads may extend along the platen or pad to the side thereof to make contact with the leads of the member, or leads extend from the electrodes vertically downward in the pad to provide signals to the processor(s) via electrical contacts (or leads) under the pad upon the platen. Further, a pad upon the platen may be of anisotropic material and when finger(s) are provided onto the pad signals travel vertically via the anisotropic material to electrodes on or in the platen. Optionally, the electrodes and/or their electrical leads are opaque to the imaging system in the scanner, but since they are sized smaller than the resolution of the imaging system in the scanner they appear invisible in an image of fingerprint(s) when captured by such imaging system.

The electronics of the scanner determines one or more liveness parameters in accordance with signals received from electrodes, and then determines liveness of the subject in accordance with one or more of (i) the presence of the such liveness parameter(s), (ii) the liveness parameter(s) when compared to range(s) for such parameter(s) associated with a typical living human, or (iii) the liveness parameter(s) as being in a range for the particular subject (based on data for such parameter measured previously and stored in memory of a computer system coupled to the scanner), or other biometrics, such as age or gender associated with the subject in such memory. The liveness parameter(s) represent one or more of skin impedance, skin temperature, or heart pulse of the subject, but other parameters for detecting liveness of a subject scanned may be used. The electronics may utilize electrodes to both transmit and receive electrical signals and information from the image of the fingerprint from the scanner may be used to select which electrode(s) to use for liveness detection. Measured impedance may include the real skin impedance values, the imaginary impedance values, resistivity, capacitance, induction, or any combination thereof. Preferably, liveness parameters represent at least complex impedance values calculated as a function of electrical frequency and then compared with the typical impedance ranges for skin.

Electrical liveness analysis may be conducted on the entire finger(s) or skin presented to the scanner or may be conducted on only a portion of the skin presented, by way of example, the fingertips. The electronics may determine liveness of each finger in accordance with electrical signals from all or selected ones of electrodes associated with the location in the image of each finger. The one or more fingers may be in contact with the electrodes of the platen disposed beneath such fingers, which may be composed of an image enhancing material, or the one or more fingers may be in non-contact with the electrodes via an image enhancing material layer that may be fixed or removable, or one or more fingers may be spaced by a gap from the electrodes of the platen.

Preferably, when electrodes are upon the platen, either on the platen surface or in a pad, the electrodes are covered by at least an upper layer of image enhancing material to provide optimal contact with the skin of the fingers and thus improve imaging of ridges and valleys of fingerprints. To provide such optical contact, a pad applied to the platen surface (or the platen surface itself) may have a porous material providing one of liquid or gel material in which only ridges of fingers are wet by the liquid or gel material when the fingers are in contact with the pad. The image enhancing material is optically transparent to the imaging system, and may be tacky or non-tacky to the touch of fingers. The electrodes are also preferably optically transparent to the imaging system in the scanner. The porous material and the liquid or gel material are close in index of refraction, and the porous material may have a fine porosity that is below the resolution of the imaging system of the scanner, such that image by the scanner is not negatively effected by the presence of these materials to improve optical imaging of fingerprint(s) by the scanner.

In one embodiment of the platen or the pad, the electrodes have leads extending from each electrode, and an insulating material over at least the leads with openings for the electrodes such that fingers can contact the electrodes via such opening when fingers are presented to the platen or pad, or the insulating material covers both the electrodes and their leads. The insulating material may be substantially thicker over the leads than the electrodes when applied over both the leads and electrodes such that more insulating material is present for the leads. Also to provide more insulating material over the leads than the electrode, the electrodes may be disposed upon insulating material while their leads downwardly extend along the platen or pad at a lower level than the electrodes in the insulating material. The electrode may also be disposed at different heights, if needed. The insulating material may optionally also be an image enhancing material, as describe earlier.

The present invention also embodies a method for imaging fingerprints having the steps of presenting a plurality of fingers to a platen having a plurality of electrodes in or upon the platen, capturing an image of fingerprints of the fingers presented, and detecting liveness of such fingers in accordance with electrical signals provided from the electrodes. When the electrodes are upon the platen, the method further has the step of providing a pad upon the platen having the electrodes and the fingers are presented to the platen via contact with the pad. The detecting step is carried out in accordance with signals representing one or more of real skin impedance, imaginary impedance, skin temperature of the subject, heart pulse of the subject, skin resistivity, capacitance, or induction. A layer may be provided over the platen and electrodes of material which enhances the image of a fingerprint from each finger by the capturing step. The fingers may be in contact with the pad or platen, or spaced by a gap from the electrodes when the liveness detecting step is carried out.

Thus, the apparatus, such as a scanner, and method of capturing skin topology determines if the skin topology presented is real or a fake finger(s) and whether or not the real finger(s) is live or dead. For the purposes of describing the invention, skin topology is referred to as being that of a human fingerprint, although the invention may be applied to other areas of skin topology such as that found on a palm, foot, or face and may be human or mammal. The capture of an image of the skin topology may preferentially be captured optically, but may also be captured via ultrasound. Specifically the apparatus distinguishes a real fingerprint from a spoof based upon electrical readings made of the skin of the finger(s). The apparatus measures electrical properties of the skin presented to it that may include resistance, conductivity, capacitance, induction, or any combination thereof, heretofore termed impedance, and determines if the electrical readings fall within the range of live human skin or determines if these readings match to within a certain threshold the electrical readings of the subject taken during enrollment or during any one or more of the previous times the subject was identified by the apparatus. Preferably, the apparatus is capable of determining the electrical characteristics of not only the outer surface or epidermis of the skin, but also the electrical characteristics of the skin below the outer surface, such as the dermal layer. The impedance of the skin may be analyzed at a range of frequencies, which may include a direct current (DC) reading. By analyzing the skin at multiple frequencies, it becomes more difficult to fake the apparatus since the impedance at multiple electrical frequencies must be mimicked by presented spoof finger(s). Probe signals of multiple frequencies may be created by having separate electrode(s) may each emit a unique electrical probe frequency, or preferably multiple probe signal frequencies, which may be emitted sequentially or in parallel from a single electrode. To emit in parallel multiple electric frequencies from a single electrode, the electrode emits a non-sinusoidal electric pulse whose Fourier decomposition contains a range of electrical frequencies. By way of example, a non-50% duty cycle square wave of fundamental frequency ω₀ may be used as this wave contains higher-order Fourier harmonic signals of frequencies 2ω₀, ω₀, etc. In order to make it difficult for people intending to spoof the apparatus to learn what frequencies are analyzed, the electrodes may randomly change the frequencies and/or pulse shapes they emit at.

The electrical properties measured may also include voltage for the determination of the subject being a live human. The heart generates a series of electrical voltages as it beats and these can be detected by having a subject touch a platen of the apparatus or can be detected remotely with no contact to the subject. The electrical heart signals detected can be used for a simple liveness check (e.g., compared versus the typical temporal voltage profile of humans) or can be used as a metric for matching to a specific person as the heartbeat profile of a subject will differ from person to person. The match of the heartbeat profile may be fused with the match score of the fingerprint in order arrive at an overall match score of the subject to the records stored in an identification database. Similarly the fusion score may incorporate readings from a temperature measuring device (optical or electrical) that is incorporated into the apparatus, where the device measures the ambient temperature as well as the temperature of the skin presented to the apparatus.

For the case of the apparatus capturing optical images of the fingerprint, it is preferable, though not required, that these electrodes are optically transparent as it is preferable that the sensing of whether or not a fingerprint is real or a spoof is conducted in the same area where the image of the fingerprint is captured. In one example, the electrodes of the apparatus may be made of indium tin oxide (ITO), an optically transparent electrically conductive film. A pure ITO film is typically deposited in a vacuum deposition process, but nanoparticles of ITO may be mixed with a second material (e.g., silicone) allowing conductive electrodes to be formed without a vacuum deposition process. Such mixing methods include combining the ITO with silicone or similar optical transparent gel-type material in sufficient percentage to allow for electrical conduction. The mixed solution allows for the screen-printing, casting, or molding of transparent or translucent electrodes. In a second example, the electrodes are deposited using a conductive ink (typically metal particles linked in a suspending matrix) where these conductive inks may or may not be optically transparent. In a third example, the electrodes are made from a metal where the metal may be opaque, but may alternatively be thin enough that at the wavelength the apparatus images the fingerprint, the metal electrodes are sufficiently transparent to view a fingerprint. Gold, for example is a metal that can be deposited very thin (sub-micron) and can transmit a portion of the incident light that is in the blue and UV portions of the electromagnetic spectrum. Similarly for non-optical imaging of the fingerprint, for example ultrasound, the electrodes preferentially provide minimal impact on the imaging capability of the apparatus.

The electrodes may be arranged in order to accommodate a single finger, but are preferentially arranged in order to capture multiple fingers. To accommodate multiple fingers, electrodes may be arranged such that fingers must be placed in relatively specific locations on a platen. The subject can be guided to place their fingers in these specific locations via mechanical means (e.g., plastic molded dividers that separate the individual fingers) or via visual means (e.g., outlines or tinted areas on the platen indicating where fingers should be placed above). However, it is preferable that the apparatus does not require the subject to be concerned with how his or her fingers align with a given electrode pattern on the platen and therefore electrode patterns that can accommodate a wide range of finger placements. Such preferred electrode arrangements include the use of long bars and small rectangular electrodes in specific array patterns that fill the finger capture platen area. By comparing the image of the fingerprint to the known areas in the image that the electrodes are sensing, one can determine whether the fingerprint may completely cover, partially cover, or miss a given electrode. By segmenting the fingerprint image (i.e., parsing out the areas of the 2D image that contain actual fingerprints from those areas where the person's fingers did not get captured), the electrode contact area can be obtained and this calculation used to determine the electrical response of the fingerprint. Since a fingerprint is composed of a series of ridges and valleys, generally within a given fingerprint region, only a fraction (for example 25 to 75%) of that area is truly making contact with the platen. This ridge-valley contact information obtained from the 2D image of the platen allows for more precise information regarding the electrode coverage area that can be used in the calculation of the electrical response of the fingerprint.

The electrodes of the platen may be in direct contact with the skin, such that direct current (DC) resistivity of the skin may be measured as well as alternating current (AC) values for resistance, conductivity, induction, or impedance may be measured. Preferably, the platen is composed of material, with electrodes within or on the surface of the platen, in which such material improves the ability of the fingerprint capture apparatus to capture fingerprint images. The image-enhancing material may be affixed to the platen of the apparatus so as to be permanent or removable. If the attachment is permanent, this may be achieved through the use of an adhesive (e.g., optically clear epoxy) or because the material itself in the coating process bonds to the platen material or through the use of an adhesion-promotion layer(s) that is first deposited onto the platen. If the image-enhancing material is removable (referred to as a pad), then this is advantageous for high-traffic applications where the image-enhancing material can be damaged and requires replacement. For optical fingerprint scanners (or imaging systems), by way of example, the image-enhancing material or pad may be composed of a low surface energy material, such as silicone or urethane, that enables dry skin to “wet” or in other words make good optical contact with the pad. In another example, the image-enhancing material or pad may be composed of a porous material that contains at least one second material that is a liquid or gel that allows for the wetting of the finger skin to the pad. The advantage of the “wetting” properties of the material is not restricted to optical imaging applications, but may also be optimized to improve imaging using other technologies such as ultrasound by facilitating the ultrasonic coupling of the platen to the skin of the finger. If the fingerprint capture is achieved optically, it is preferable that the electrodes are sufficiently optically transparent to enable the capture of the fingerprint image through the electrodes.

The electrodes when present on the image-enhancing material of the platen attach to the appropriate electrical contact points on the rest of the apparatus to allow for the sensing of the electrical properties of the object that comes in contact with the platen. For the case of the platen being a removable pad, the electrical contacts of the pad may make electrical contact with electrical leads of the apparatus in several ways. In one embodiment, the pad is held in place by a mechanical clamp and the clamp has the required electrical leads or conduits to send signals from the interior of the apparatus to the pad and vice versa. In another embodiment, no mechanical clamp is required, but the pad itself has the electrical leads that enable electrical signals to conduct from the top of the pad to the bottom of the pad where at the bottom of the pad the electrical leads make contact with leads on the platen and the pad is placed such that electrical signals can propagate from the inside of the apparatus to the pad and vice versa. In a third embodiment, the pad is composed of anisotropic conductors that conduct electricity primarily perpendicular to the pad's fingerprint surface, thereby transmitting the electrical signals to the pad from the surface it rests on and vice-versa without the need for electrodes to be patterned on the pad itself.

Alternatively, the electrodes of the platen are not in direct contact with the skin, and are separated by a small gap provided by a layer of the above described image-enhancing material that aids in the image capture of the fingerprint. The finger thus is in direct contact with this additional image enhancing material. This image-enhancing material may be deposited directly to the platen's electrodes or otherwise deposited on a layer or layers that are built on top of the electrodes and as such the material is a permanent fixture to the fingerprint capture apparatus. The material may also be in the form of a pad that is placed on the platen surface to improve the ability of the device to capture a fingerprint image, where the pad is removable. As stated earlier, removability of the pad is advantageous for high-traffic applications where the image-enhancing material can be damaged and requires replacement. In a further alternative, the finger need not be in physical contact with the apparatus, but is disposed at a gap above the apparatus. In this case, the image enhancing material need not be present on the platen, but it may desirable to have a material on the platen, such as to protect the platen's electrodes. The electrical properties of the skin are detected at a distance (i.e., remotely) and therefore AC signals are solely used. In one example, the apparatus may capture a touchless fingerprint wherein the finger does not make physical contact with any portion of the apparatus. Preferably the detection of finger electrical properties, such as impedance or the periodic voltage changes associated with a heartbeat are focused to fingers using the image captured by the scanner of such fingers to select which electrodes to use and/or how to electrically address the electrodes such that the desired portion of the finger is analyzed for liveness.

For the electrodes and electronics associated with the capture of electrical signals from a human, noise reduction and signal amplification may be desirable. Where the skin is in direct contact with the electrodes, the electrical signals will be the strongest, but there will be ambient electrical noise of 60 Hz and higher order harmonics that must be filtered. This is also the case when the skin is not in direct contact with the electrodes and particularly when the skin is an appreciable distance away from the electrodes. As a consequence, the electronics may incorporate differential measurements, common mode rejection, guarding, negative feedback or similar noise cancellation techniques to improve signal detection from electrodes. Likewise, the electrodes are preferentially large to maximize the signal, and the impedance of the electrodes and the leads themselves kept low through proper material selection.

In another embodiment, the fingerprint platen contains at least one electrode, in which the platen is split into two platen areas, where each platen area has at least one electrode. The subject places at least one finger of a separate hand on each of the platen areas. The electronics of the apparatus detect an electrical response of the fingers through the heart of the subject. In this manner, in addition to the apparatus measuring electrical impedance or other parameter(s) of the subject, the subject's heart beat may also be measured to provide another liveness parameter.

### DETAILED DESCRIPTION OF THE DRAWINGS

The foregoing objects, features and advantages of the invention will become more apparent from a reading of the following description in connection with the accompanying drawings, in which:

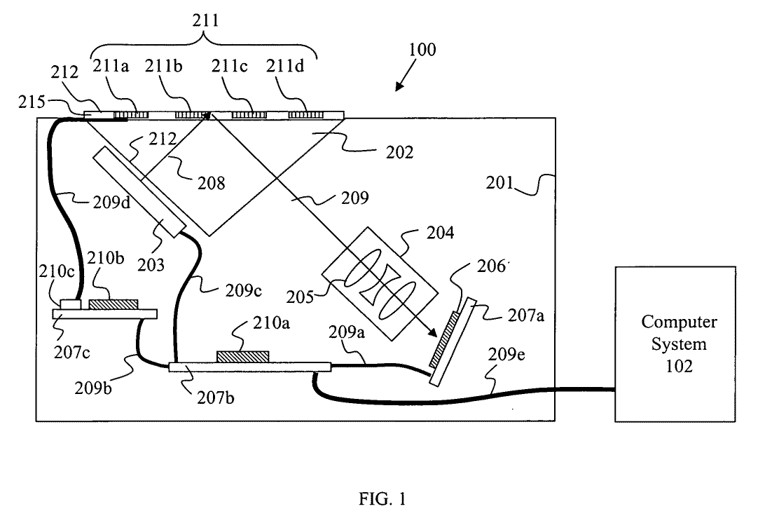

FIG. 1 is a block diagram of a fingerprint scanner or image capture apparatus having an optical imaging system to capture images of the skin presented to a platen which incorporates electrodes of the present invention for liveness detection;

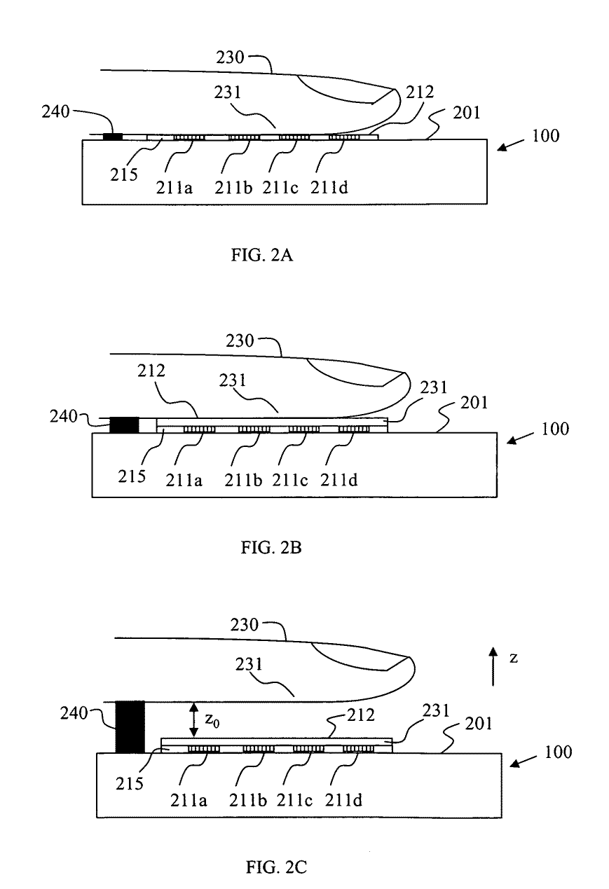

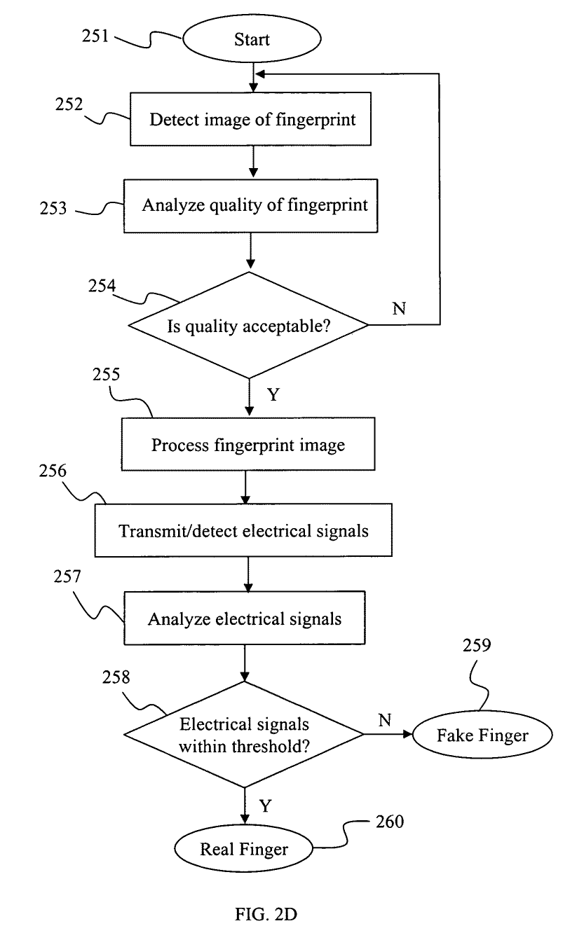

FIGS. 2A, 2B, and 2C are block diagrams of FIG. 1 showing in the more detail examples of the electrodes and the relative placement of a finger and associated fingerprint with the electrodes in three different configurations, in which the components in the housing of the apparatus are not shown for purposes of illustration;

FIG. 2D is a flow chart of the process for capturing and analyzing an image of a fingerprint and the transmission, detection, and analysis of electrical signals for determining liveness in the apparatus of FIG. 1;

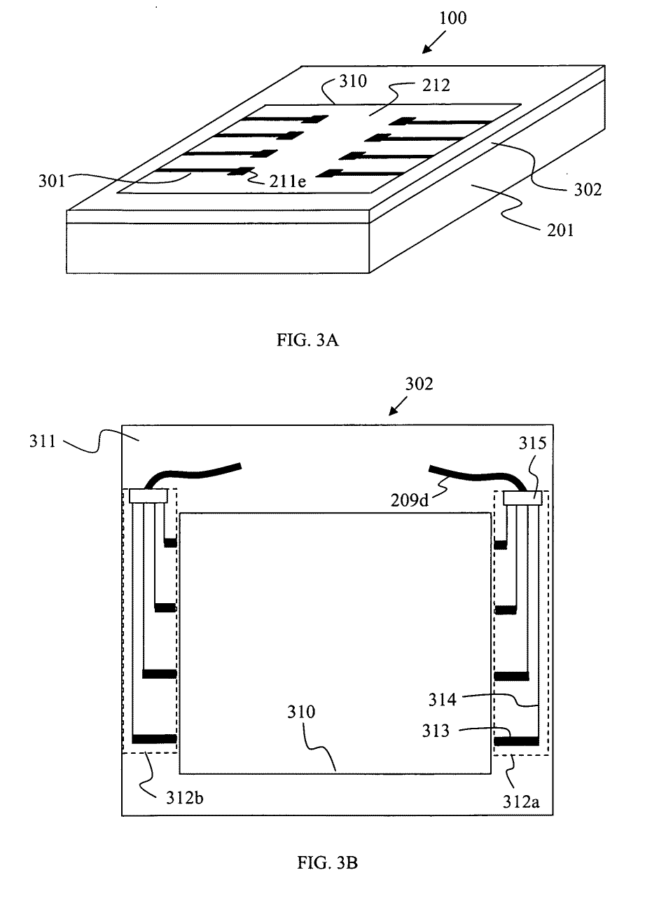

FIG. 3A is a perspective view of the apparatus of FIG. 1 showing a platen for a single finger;

FIG. 3B is a bottom view of the cover of the apparatus housing of FIG. 3A showing electrical leads;

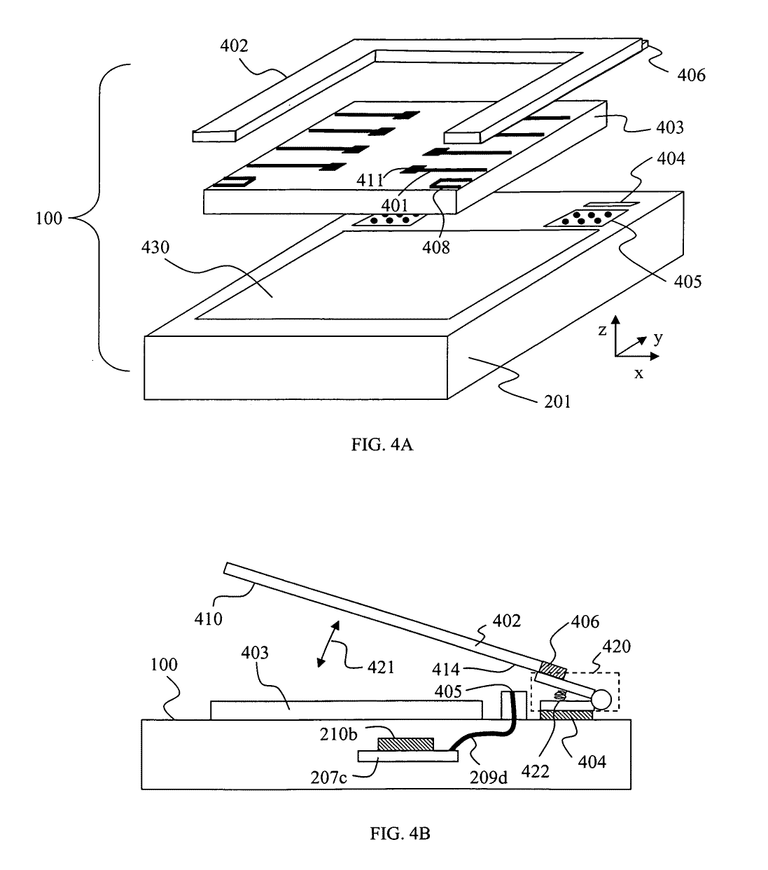

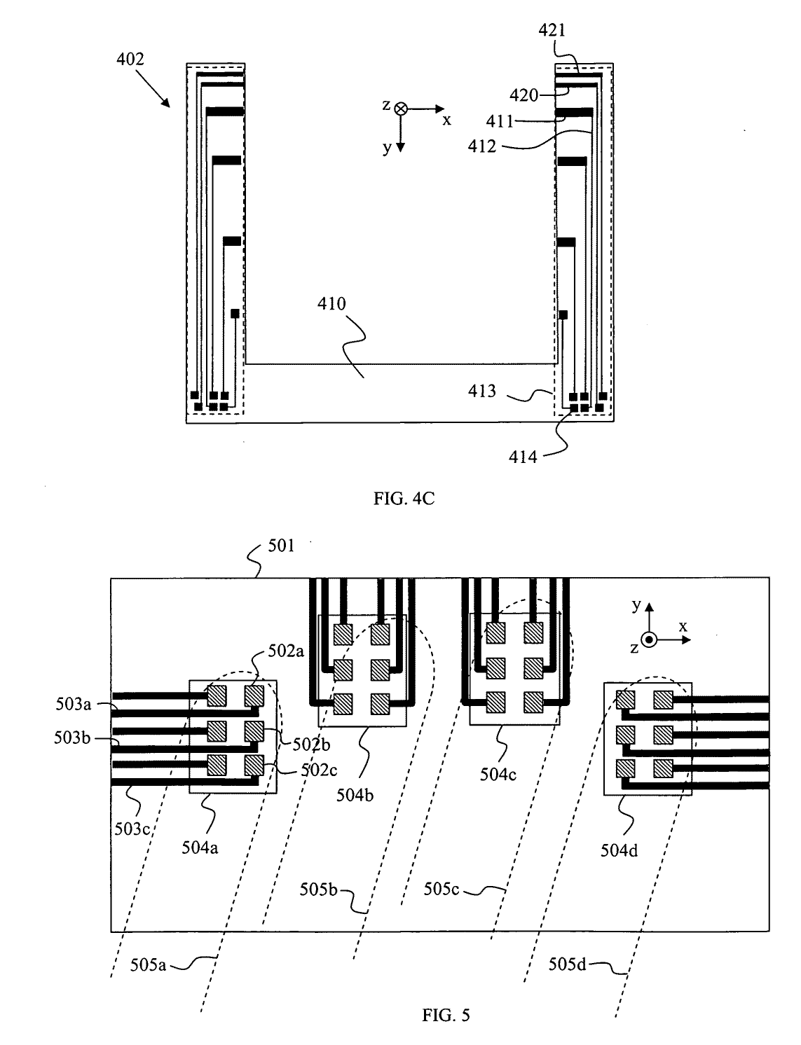

FIG. 4A is a exploded perspective view of the apparatus of FIG. 1 showing the platen of the apparatus as a removable pad which is assembled between a clamp and the apparatus housing;

FIG. 4B is a side view of the apparatus of FIG. 4A in which the clamp is shown pivoted with respect to the apparatus housing;

FIG. 4C is a bottom view of the electrical leads of the clamp of the apparatus of FIGS. 4A and 4B for connecting the electrodes of the pad of FIG. 4A with the processor(s) of FIG. 4B for to detect liveness of a finger;

FIG. 5 is a top view of another embodiment of the electrodes of the platen in the apparatus of FIG. 1 for placement of multiple fingers which requires specific finger placement in order to detect liveness, in which fingers are shown by dashed lines;

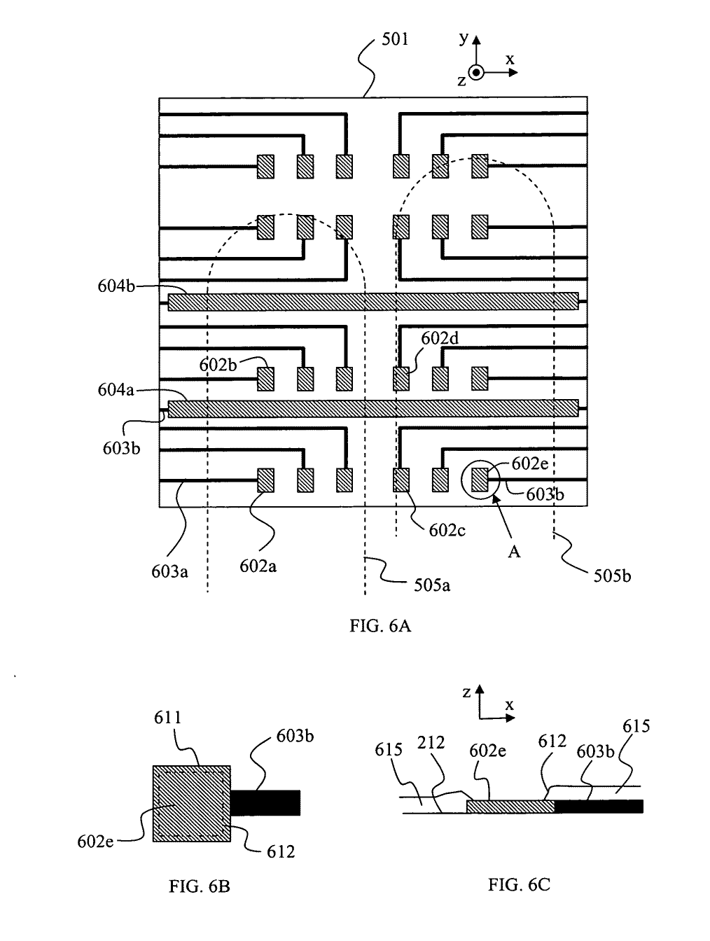

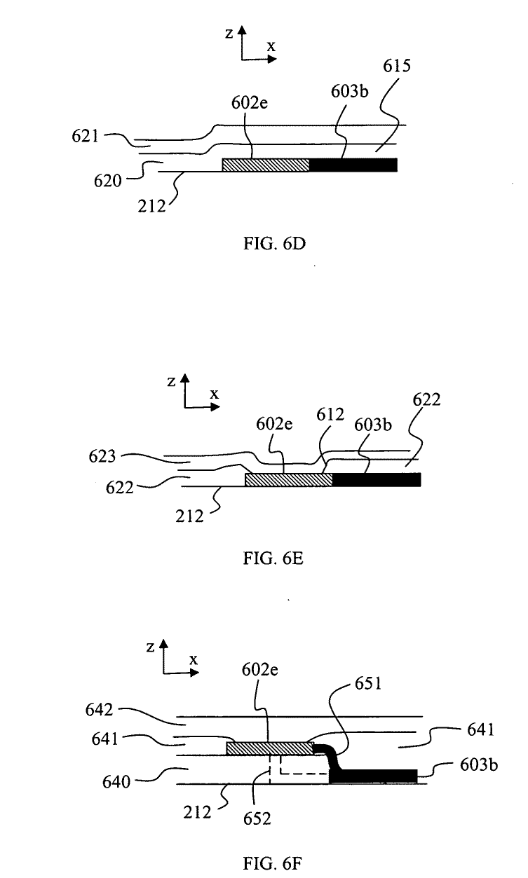

FIG. 6A is a top view of still another embodiment of the electrodes of the platen in the apparatus of FIG. 1 for placement of multiple fingers showing electrodes in a layout that does not require specific placement of multiple fingers in order to detect liveness, in which fingers are shown by dashed lines;

FIG. 6B is a more detailed view of one of the electrodes of FIG. 6A

FIG. 6C is a cross-section of the electrode layout of FIG. 6B that illustrates one embodiment of patterned coatings for electrodes and to insulate electrical leads;

FIG. 6D is a cross-section of the electrode layout of FIG. 6B that illustrates another embodiment where the insulating material is provide over both electrodes and their electrical lead with an optional image enhancement layer;

FIG. 6E is a cross-section of the electrode layout of FIG. 6B that illustrates a further embodiment where the insulating material over the electrodes and electrical leads are such that there is thicker total coating over the electrical leads than over the electrode;

FIG. 6F is a cross-section of the electrode layout of FIG. 6A that illustrates still another embodiment where the electrodes but not the electrical leads are patterned on top of a material and then optionally overcoated such that the electrodes are closer to the skin as compared to the electrical leads;

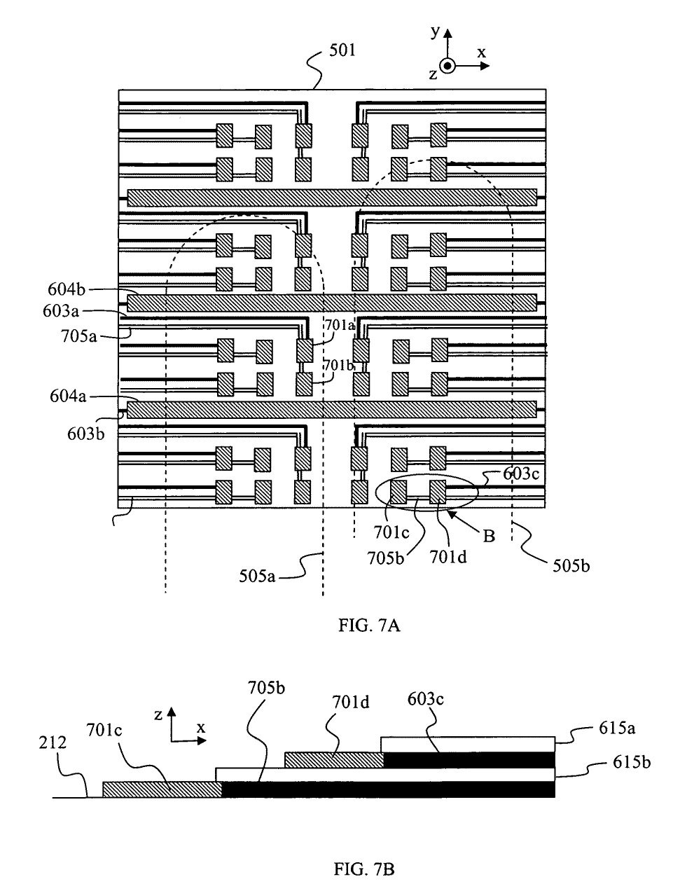

FIG. 7A is top view of the electrodes of the platen in the apparatus of FIG. 1 in accordance with a further embodiment for placement of multiple fingers showing an electrode layout that does not require specific placement of multiple fingers in order to detect liveness and which incorporates more than one level of electrodes, in which fingers are shown by dashed lines;

FIG. 7B is a cross-section of the electrode layout of FIG. 7A illustrating multiple patterned coatings for different levels of electrodes and electrical leads;

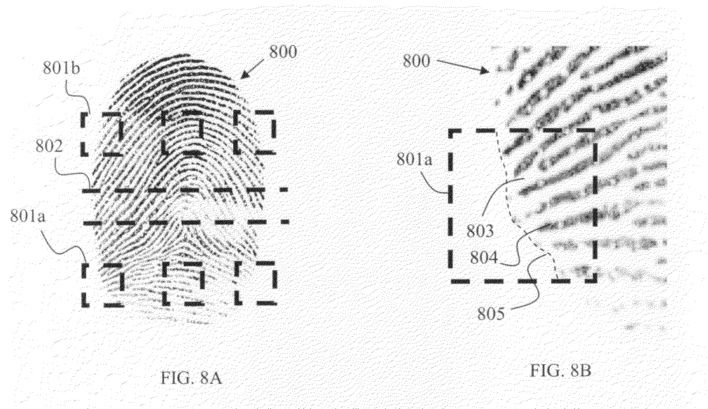

FIG. 8A is an example of part of an image one of the fingerprint captured by the apparatus of FIG. 1 using the platen of electrodes of FIG. 7A in which certain of the electrodes are shown in dashed lines to indicate the overlay on the areas sampled by such electrodes and the ridge contact area of the image can be used to calculate the area of contact on the electrode sampled regions;

FIG. 8B is a more detailed view of a portion the image of FIG. 8A;

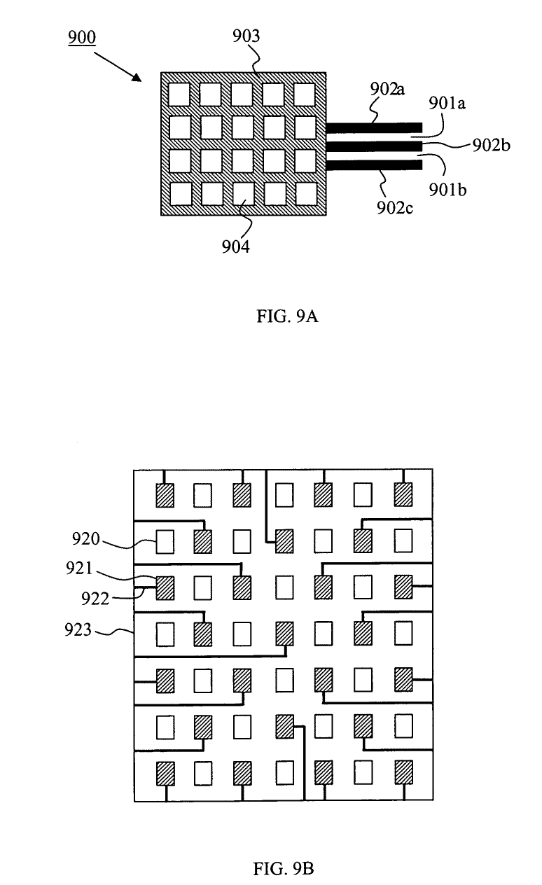

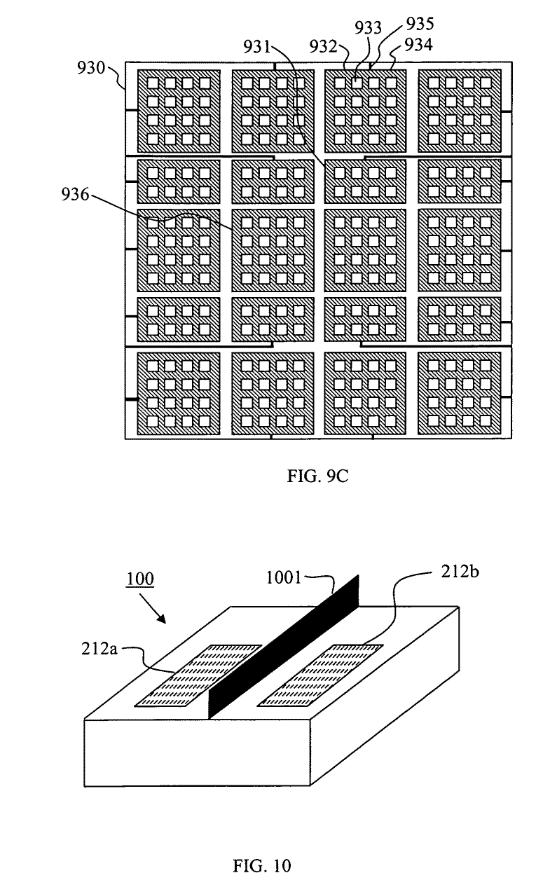

FIG. 9A is an example of another electrode that may be used in place of the electrode shown in FIG. 6B having both conductive and non-conductive areas;

FIGS. 9B and 9C are other electrode layouts or patterns with the electrodes and their leads smaller than the resolution of the imaging system; and

FIG. 10 is an isometric view of a fingerprint capture device in which the finger(s) from two separate hands of a subject enables measurement of electrical signals through the subject's heart.

### DETAILED DESCRIPTION OF THE INVENTION

Referring to FIG. 1, an apparatus 100 for capturing fingerprints for one finger or multiple fingers of a hand is shown. Multiple electrodes are provided in or upon a platen 212 located on a housing 201 in which the platen is sized for capture of an image of one or a desired number of fingers. Housing 201 has an illumination source 203 for sending light through one facet 212 of a prism 202 that is substantially transparent at the wavelength of light source operation. Light (denoted by ray 208) from the illumination source strikes platen 212 and reflected light (denoted as ray 209) then propagates through an objective lens 204 (composed of one or more optical imaging elements 205) which focuses the reflected light onto two-dimensional (2-D) sensor or detector 206. The reflected light represents an image of the surface topology of the skin or the finger(s) presented to platen 212 at or about the fingertips as typical of fingerprints. However, the present invention is not restricted to the optical and/or electrical sensing of skin topology comprising the fingertips of one or more finger, but may be extended to any skin topology of a human or mammal. Sensor 206, by way of example, may be a complementary metal-oxide semiconductor (CMOS) or charge-coupled device (CCD) sensor that is mounted to a circuit board 207a and connected, via power and communication cable 209a, to a main circuit board 207b of the apparatus 100. The main circuit board 207b contains electronics having at least one or more processors 210a that allow it to control the sensor 206, via cable 209a, and the illumination source 203, via cable 209c, as well as performing image processing, if needed, of the image captured and received from sensor 206 of the fingerprint(s). The processor(s) 210a send the captured image to an external computer system 102 via a power and communication cable 209e or may be wirelessly connected. The computer system may optionally be integrated into the same unit or housing as apparatus 100.

Multiple electrodes 211 are incorporated in the surface of platen 212, such as illustrated by electrodes 211a, 211b, 211c, and 211d. These electrodes are located along the platen surface in the areas according to where the single finger or each of the multiple fingers of a subject are disposed against or adjacent to the platen's upper surface. These electrodes will be described later in more detail in connection with FIGS. 4A-9. Electrodes 211 sense the electrical signals, e.g., analog voltages, current, or capacitance, provided by fingers disposed against or adjacent thereto, and such signals are sent via cables or wires 209d to electronics on spoof-detection circuit board 207c for processing by one or more processors 210b, via amplification and/or noise reduction electronics or circuitry 210c. Although referred as electrodes, since the electrodes are electrical components that transmit as well as receive, as well as sense the electrical properties of skin at a given separation distance, it is understood that they may also be termed and function as antennae, transducers, receivers, thermocouples, and electrical dipoles to the electronics utilizing such electrodes for liveness detection.

Electronics 210c may in addition to improving quality of detected signals from one or more electrodes 211 by amplification and/or noise reduction, if needed, may also incorporate electronic functionality for generating and transmitting electrical signals of different amplitudes and frequencies (for example DC to 100 MHz) to one or more electrodes 211 via cables or wires 209d, or other signal generator means under control of processor(s) 210b may be used providing a frequency and/or amplitude tunable DC or AC signal source. Electrodes may be present to both send and receive signals, or different ones of electrodes 211 may be used for sending and receiving signals. Processor(s) 210b may further be able to select which of electrode(s) 211 are selected to send and/or receive signals. The platen 212 containing the electrodes 211 may be an integral part of prism 202 or may be a separate material that may be affixed to the prism 202 such as with an optical adhesive or may be a removable component (such as a pad or sticker) that is placed on the prism to facilitate the electrical tests conducted by the apparatus. The processor(s) 210b are programmed in accordance with software to analyze the signals received from one, all, or combinations of electrodes 211 to determine (or detect) one or more liveness parameters, as described later below.

In FIG. 1 the fingerprint imaging operates by bright-field illumination optical total internal reflection (TIR), such as is used commonly in optical fingerprint devices (see for example U.S. Pat. No. 3,174,414), however other means for imaging finger prints may be used in housing 201, such as optical imaging system operating in accordance with dark-field optical illumination (e.g., U.S. Pat. No. 5,233,404), touchless optical imaging (e.g., U.S. Pat. No. 6,853,444), non-TIR imaging (e.g., U.S. Pat. Nos. 3,138,059 and 7,147,153), or ultrasonic imaging (e.g., U.S. Pat. Nos. 6,296,610 and 7,236,616). The spoof detection described herein may be used with any fingerprint imaging means that occurs at or about the same time when fingerprint(s) are imaged along platen 215.

The particular processing by electronics for liveness detection on spoof detection board 207c may be in accordance with methods for such purpose as described below for one or more of measuring the impedance values or heartbeat (or rate) from electrodes, are not limited to those described herein. The particular liveness parameter(s) determined are compared to value(s) stored in memory of the processor(s) 210b representing ranges or values for the general human population to determine whether or not finger(s) lying against or adjacent to the platen 212 at or about the time of imaging onto sensor 206 is real or fake. The computer system 102 is sent the result of the liveness check with the associated image captured via board 207b. The operator of the apparatus 100 or security personal are then notified if the liveness parameters are outside predefined ranges and thus spoof attempt has been detected. Optionally, such comparison is performed by computer system 102, which receives detected signals for determining the liveness parameter, via processor(s) 210b, such as via cable 209b to board 207b. Also optionally, the one or more processors 210b may not be provided and all processing provided by computer system 102.

The detection and processing of the signals from electrodes may be as described in A. J. Hinton and B. Sayers, “Advanced Instrumentation for Bioimpendance Measurements,” 1998. Hinton and Sayer describe an electronic apparatus marketed by Solartron Analytical (Farnborough, Hampshire UK) for the purposes of electrically probing human skin and tissue. The device employs a 4-electrode system capable of analyzing skin tissue with a range of electrical frequencies from 100 Hz to 1 MHz. Hinton and Sayer specifically cite the example of looking inside of skin at organs and detecting electrically if they are live or dead, similar to the application discussed here where the liveness of the skin presented to the apparatus is the question. U.S. Patent Application 2005/0281441 appears to also describe the use of a four-point measurement of complex impedance to analyze the electrical properties. Also, the detection and processing of signals from electrodes may provide electrical impedance tomography (EIT). EIT is described for example in Henderson R. P. and Webster J. G. (1978) “An Impedance Camera for Spatially Specific Measurements of the Thorax,” IEEE Trans. Biomed. Eng. 25: 250-254). In EIT the area of the body to be analyzed electrically is attached to a series of electrodes and then alternating current, below the level that would be required to stimulate nerves, is passed through a series of electrodes where the current can alternate between different electrode pairs and the frequency of the signal can change. The magnitude and the phase of the AC signals are analyzed by a computer in order to map the tissue beneath the skin. A detailed description of the signal processing and the electrical circuitry required for EIT may be found in Electrical Impedance Tomography: Methods, History, and Applications, ed. D. S. Holder (CRC Press, 2005). Further, the detection and processing of signals from electrodes may sense conductivity changes in the skin, such as used for remote sensing of ground terrain for conductivity changes, to detect finger liveness. Such electromagnetic (EM) remote sensing has been used to map salt water and to spot fresh-water springs and has been used to map ground terrain to detect objects within the earth, using handheld, airplane, and satellite mounted devices. For example Stratascan (Worcestershire. UK) performs land scans and examples of mapping terrestrial EM variations from air or space are Allen D A and Merrick N P, 2005, “Surface water/groundwater interaction investigation using a towed geo-electric array” Conference Proceedings. Irrigation Association of Australia, and V. K. Choubey, “Monitoring surface water conductivity with Indian remote sensing satellite data: a case study from central India,” Hydrological, Chemical and Biological Processes of Transformation and Transport of Contaminants in Aquatic Environments (Proceedings of the Rostov-on-Don Symposium, May 1993). IAHS Publ. no. 219, pp. 317-326 (1994). Also, detection and processing of electrical signals from the electrodes may as described in U.S. Published Patent Application No. 2006/0058694 for the purposes of detecting a human heartbeat with electrodes/antennae that are up to 1 meter away from the subject. Since the voltage produced by the human heart is weak and there is significant background noise such as 60 Hz and its higher frequency harmonics due to electrical outlets and light fixtures, this published application discusses different electronics for improving the detection of the desired signal and to suppress unwanted noise (such as may be incorporated in electronics or circuitry 210b). Electronic approaches include the use of positive feedback, such as guarding, bootstrapping, and neutralization. This published application also discusses the use of negative feedback techniques to ensure that the positive feedback techniques do not become electrically unstable. By incorporating by reference herein the electronics of this U.S. Published Patent Application No. 2006/0058694 in apparatus 100, the electronics of the present invention are able to detect electrical signals remotely for the purpose of liveness detection, but other means for electrical signal detection using electrodes 211 may also be used.

For electrodes 211 that are in contact with the skin, the electronics using such electrodes 211 may be as published by Techniloc Technologies in its Model K-7 Technical Maintenance Manual for liveness detection. Such electronics have been used by Cross Match Technologies (Palm Beach Gardens, Fla.) in a modified version of its Verifier 250 for liveness detection of a single finger in 1999 through 2000. As described by the Techniloc manual, an electrical schematic of the circuit and the multiple electrodes used to detect the impedance of the finger may be employed in apparatus 100.

FIGS. 2A-C illustrate how in the case of a single finger a finger may be disposed relative to platen 212. However, platen 212 may be similarly structured for accommodating spoof detection for multiple fingers when captured by apparatus 100. In FIG. 2A, the platen 212 is patterned with electrodes 211 (illustrated for example by electrodes 211a through 211d) and the finger 230 with its fingerprint 231 is in direct contact with such electrodes. The direct current (DC) impedance and the alternate current (AC) impedance at a range of electrical frequencies of the fingerprint skin may be detected and measured by processor(s) 210b using signals from such electrodes. The measured AC impedance is then compared to that of a general human or that of a database of specific humans whose electrical properties were recorded during an enrollment procedure by the computer system 102 having memory storing such database. Platen 212 has a layer of material 215 containing electrodes 211 which may be the same material as that of prism 202, and as such an integral part of the prism or it may be a separate material that is added to the prism surface. Preferably material 215 is an image-enhancing material which facilitates the optical or ultrasonic coupling between the skin and the platen, depending on the type of imaging operative in housing 102. By way of example, for an optical system, such image-enhancing material may be silicone, urethane, or similar low surface energy material (such as described for example in U.S. Pat. Nos. 6,259,108 and 7,319,565). Although illustrated schematically as an arrangement of two materials (materials 215 and electrodes 211), the platen may include several layers of materials, particularly for the case of a pad that is removed and replaced in the field, wherein such pad may be composed of more than one layer of silicone or urethane with a stiffener sheet composed by way of example of polycarbonate or acrylic between them as described in U.S. Pat. No. 7,319,565. Although many silicones and urethanes that achieve excellent dry skin image enhancement tend to feel tacky to the touch of skin, non-tacky image-enhancing material may be utilized as described in U.S. Provisional Application No. 61/271,903 which is herein incorporated herein by reference. As detailed in this U.S. Provisional Application, a non-tacky material meant for the skin to directly touch may be applied to an underlying compliant and potentially tacky material and achieve image enhancement. The non-tacky top material may be conductive in order to apply the invention described herein. By way of example, if material 215 is silicone or urethane the electrodes 211 can be integrated into such material by molding a patterned conductive material at the time that the silicone is cured. The silicone itself in selective regions may be doped with a conductive material such as metals, ITO, or semiconductors [see, for example, C. A González-Correa et al, “Resistivity changes in conductive silicone sheets under stretching,” Physiol. Meas. 23 183-188 (2002), or conductive silicone products by Dow Corning, www.dowcorning.com]. Alternatively, the silicone material, after being cured, may be patterned with conductive material through a variety of processes including silk screening and vacuum deposition. Conductive material includes conductive inks that are semi-transparent (see for example, those from Creative Materials (Tyngsboro, Mass.) as well as conductive plastics such as polyaniline, PEDOT, polyaniline, polythiophenes, and BESB. In general, conductive materials tend to be not as optically transparent as materials that are non-electrically conductive. To compensate, the areas of platen material 215 that are not made conductive may be patterned, doped or otherwise made to have a similar amount of optical absorption, such that the camera (combination of optical imaging system 204 and sensor 206) sees a reasonably uniform amount of light that comes from the illumination source 203. Also, a thin film layer (or other reflective material) may be provided along the entire platen or pad, or just underneath the conductive material of the electrodes and their leads, to achieve the same or substantially the same reflectivity as non-conductive regions (without electrodes or leads) along the platen or pad. The degree to which the field must be made uniform depends upon the image uniformity desired and pixel SNR of the sensor 206. For example, if the sensor 206 is capable of outputting 10 bits of useable signal per pixel (e.g., 2¹⁰=1024 grayscale values) and the desired uniformity must be 1% with a signal range of 200 grayscale values, the uniformity of the field is not as important as when the sensor only outputs 8 bits of useable signal (i.e., 256 grayscale values) since more electronic field flattening in software or firmware can be performed by processor(s) 210 and/or computer system 102 with 10 bits versus 8 bits. Similarly, for ultrasonic imaging, the non-conductive regions of material 215 may be patterned with a separate material in order to match the ultrasonic absorption of the conductive regions if desired.

FIG. 2B is the same as platen 212 of FIG. 2A but with the addition of a layer of material 231 which separates material 215 with electrodes 211 from the finger 230. Material 231 may serve as a protective layer for the electrodes 211 and/or a layer that improves the capture of the fingerprint image. If material 231 is a dielectric, the DC impedance of the finger skin is difficult to measure, but the AC impedance can be measured (for example, capacitance). Since the finger skin is in contact with material 231 versus material 215 as in FIG. 2A, material 215 need not be an image-enhancing material as previously described, but may for the example of an optical fingerprint scanner 100, may be glass with transparent electrodes 211 overcoated on such glass via the sputtering of ITO. It is preferential, however, that material 231 is an image enhancing material which, for the example of an optical imaging system, may be silicone or urethane. Material 231 may be permanently affixed to material 215 or may be removable (e.g., in a pad form) in order to provide protection of the electrodes, yet enable replacement if damaged or worn. Similarly, the combination of material 215 with integrated electrodes 211 and material 231 may comprise a pad that is replaceable and not permanently affixed the apparatus. Material 231 may be an anisotropic conductor (with the conductivity axis in the vertical or z-direction), thereby allowing direct electrical contact of the skin with the electrodes. Such electrodes are disposed upon or on in the platen below the pad. In the case of material 231 being removable relative to material 215 as an entire sheet, the minimum thickness of material 231 is dictated by the mechanical properties and may be in the range of a few hundred microns to ensure the material does not fold on itself or trap air bubbles. However, if material 231 is not removable or is removable using a special solvent and reapplied with a spray, liquid roller or similar application method, material 231 may be only a fraction of a micron, on the order of a micron or tens of microns in thickness.

In FIG. 2C, the platen 212 is the same as in FIG. 2B, but electrodes 211 of platen 212 sense the electrical properties of the finger 230 at a range of distances z about a nominal distance of z₀ above the platen 212. In this embodiment, the apparatus, may or may not have a material 231 covering the electrodes 211 and said material may or may not be image enhancing. The larger the separation or gap distance z₀ is, the less the individual electrodes 211a through 211d will be individually acting to sense electrical signals from specific locations of the finger as in FIGS. 2A and 2B where the fingerprint is in contact or in near contact with the electrodes. Rather, in one embodiment of apparatus 100 in FIG. 2C which detects the fingerprint in free space, the electrodes preferentially act as a phased array so as to collectively direct an electrical signal towards a given portion of the finger and to receive an electrical signal from a specific location as well. Phased arrays operate by having an array of electrodes 211 operate as emitters where the phase of each emitter is tuned relative to that of the other emitters in order to create a coherent beam that propagates away from the emitters as a plane wave, focused, or diverging wave front, such as in radar. Such phased arrays may be as developed for GHz emitters, see for example, work performed by the University of California, San Diego where as documented by the school's media relations website, (www.jacobsschool.ucsd.edu/news/news_releases), researchers have developed a silicon chip with an 8-element, 2.2×2.3 mm, 6-18 GHz phased array as well as a silicon chip with a 16-element, 30-50 GHz, 3.2×2.6 mm chip. Alternatively, or in addition, as will be described in more detail later, electrodes 211 of apparatus 100 in the non-contact case detect the voltage signals of the finger that are originally created by the heart. The non-contact embodiment may optionally incorporate shielding (e.g., a rectangular tunnel or tube rising above platen surface 212) so that electrical signals from only the skin portion desired [e.g., a finger(s)] are detected and the rest of the subject's hand, arm, or body core. Still alternatively, or in addition, electrodes 211 are not phased but rather independent transmitter/receivers that detect the impedance of an object (such as a subject's skin) placed above them.

For liveness checking of fingers it is preferred that liveness checking is performed at the fingertips as it is the imaged fingerprints of finger tips that are used for enrollment and identification. For example, although optical fingerprint scanners have been developed that capture a 3.0″×3.2″ size area (see for example the L Scan® Guardian™ from Cross Match Technologies, Palm Beach Gardens, Fla., or the TouchPrint™ 4100 of L-1 Identity Solutions, Stamford, Conn.) and are designed for an individual to place four fingers from one hand simultaneously on the platen, once an image is captured, only the fingerprint from the fingertip is segmented and kept for enrollment or database matching purposes. Consequently, it is preferred that the liveness of the fingertip region is checked, since a person may be wearing a fingerprint-patterned polymer membrane on this region, see for example, Section 9.4 entitled Fake Finger Attacks in Handbook of Fingerprint Recognition, by D. Maltoni et al, (Springer, Boston) 2003. In the case of a finger position(s) being in contact or in near contact to the electrodes as depicted in FIGS. 2A and 2B, the electrodes that are directly under the fingertips are preferably read. For the embodiment represented by FIG. 2C, substantially all of the electrodes (antennae) of the phased array are used to transmit/receive signals from a specific location to check for liveness. This location may be varied across the subject's fingers by varying the relative phase of the electrodes, wherein the choice of the locations in space to scan are aided by analysis of an optical image of the fingerprint as well as an optional range finding device which may be incorporated in housing 102 of the apparatus, which by way of example, may be an IR range finder such as GP2D120 from Sharp Electronics Corp. that detects object in a 1.5″ to 12″ range.

As shown in FIGS. 2A, 2B, and 2C, apparatus 100 may contain optional conductive bar 240 that also makes contact with the finger 230 away from the fingertip. This conductive bar would allow for signals to be detected through the path of the finger which can be advantageous. For the embodiment depicted in FIG. 2C, the conductive bar 240 may also be used to set an approximate distance of the surface of finger 230 facing platen 212 and to therefore ensure a reasonable quality fingerprint image without resorting to an opto-mechanical autofocus mechanism. Incorporated into the bar may be a thermocouple for detecting the temperature of the finger and that of the ambient environment. Such thermocouple may operate instead or, or in addition to, the use of electrode(s) 211 to determine the temperature of the skin in relationship to the ambient temperature. Skin temperature and ambient temperature may be detected at different times by signals (representative of temperature) from the thermocouple to processor(s) 210b, and thereby provide additional liveness parameter of the subject when his or her finger(s) are scanned, so long as the ambient temperature when last recently detected (i.e., when no finger(s) were present on the platen 212 and sufficient time has past since an earlier scan to allow bar 240 to reach ambient temperature) is substantially outside the skin temperature range typical of a living human. Ambient temperature may also, or instead, be provided in the apparatus by another temperature sensor, if needed. Such temperature liveness parameter being positive when the detected temperature is in range typical of a living human, and negative when outside such range.

The fingerprint image capture portion of the apparatus may be a prism-based bright-field illumination system as depicted in FIG. 1. As described earlier, this configuration is not a limitation of the current apparatus as other optical configurations such as dark-field or touchless optical imaging systems can be employed, as well non-optical fingerprint image capture technologies such as ultrasonic scanning can be incorporated into the apparatus. For the touchless fingerprint position described in FIG. 2C, the fingerprint imaging system cannot incorporate a total internal reflection (TIR) prism based system, nor ultrasonic scanning due to the large impedance of air to ultrasonic signals. Rather, the more practical image capture technology is a touchless optical imaging system, such as is described in U.S. Pat. No. 6,853,444.

The operation of the apparatus 100 is shown in the flow chart of FIG. 2D. After the Start 251 of the process, the fingerprint capture apparatus 100 in step 252 operates illumination source 203 and captures an image of a fingerprint(s) on detector 206. Other components than 202-206 may be used in accordance with the particular optical or ultrasonic imaging means of the apparatus, as described earlier. In step 253 the hardware, firmware and/or software of processor(s) 210a and/or computer system 102 analyzes the image and in step 254 a decision regarding the quality of the image is made. If the quality is not acceptable, the apparatus captures a second fingerprint image (repeating step 252) and repeats the fingerprint analysis process. By way of example, a fingerprint image may not be of acceptable quality if the image contrast is too low, an insufficient area of the fingerprint is captured, an insufficient number of minutiae points are detected, or the fingerprint image captured is not sufficiently well aligned in relation to the areas being analyzed for electrical signals by the fingerprint capture apparatus. If the fingerprint image quality is deemed acceptable, the fingerprint image may be further processed at step 255 by processor(s) 210a and/or computer system 103, in order to aid in the electrical spoof analysis. By way of example, such processing may include the segmentation of the fingerprint region(s) from the rest of the image and the determination of the contact ridge area versus valley area of the fingerprint to determine later (step 257) what area of the fingerprint is being analyzed by the apparatus' electrodes 211.

As will be described later in more detail in connection with FIGS. 8A and 8B, memory of the processor(s) 210a or the computer system 102 may store pixel locations associated with each of the electrodes in the two dimensional image captured by the detector 206. The grayscale intensity of the pixel values along regions of pixels in a captured image associated with each electrode may be used to determine when such electrode disposed at (or near) pixel locations in the image associated with the fingerprint, and thus only signals from such electrodes may be used, if desired, for liveness detection.

In step 256, the apparatus 100 transmits and/or detects electrical signals that are generated by the finger(s) themselves via all, one, or combination of electrodes 211 along the same platen through which imaging was carried out or in parallel or before fingerprint imaging to processor(s) 210b. In the case of electrical signals generated by a finger itself, the processor(s) 210b check for the periodic voltage changes that are generated by a human heart, and in the case of receiving electrical signals due to a series of electrical signals provided and/or detected by electrodes 211, the processor(s) 210b detect the impedance of the finger (or other finger(s)) presented to the apparatus. Additionally, the temperature of skin and the ambient temperature may be sensed by providing one or more electrodes 211 operative as an electrical thermocouple (see for example, U.S. Pat. No. 3,853,383), or alternatively an optical temperature probe (such as an IR noncontact thermometer manufactured by Raytek Corp. of Santa Cruz, Calif. or Extech Instruments Corp. of Waltham, Mass.). Such thermocouple or probe may also be provided on housing 201 to contact finger(s) when present on or adjacent to platen 212, and provide signal(s) to processor(s) 210a programmed to analyze such signal(s).

As stated earlier, the detected electrical signals from electrode 211 are analyzed by the apparatus and compared to a predetermined range of electrical signals deemed to represent those of a general human or to the electrical signals captured and stored of individuals during the time of enrollment. If the detected electrical signals are within a certain threshold value of the electrical signals deemed to be from a specific person or a general human, then the finger is accepted as real, step 260. If not, the finger is declared as being fake, step 259. Alternatively the apparatus may output a fingerprint spoof score that ranks the finger, where by example a low score means the likelihood of the finger being a fake is low and a high score means that likelihood of the finger being a fake is high. In this case, the apparatus is preferentially connected to a computer system 102 that makes the determination as to whether or not to accept the finger as real or fake based upon the spoof score received from the apparatus and/or based upon additional spoof scores through other means of detecting fake fingers such as blanching (see for example U.S. Pat. No. 4,728,186) or multi-spectral imaging (see for example U.S. Pat. No. 7,147,153).

Referring to FIG. 3A, one embodiment of the array of electrodes 211 incorporated into platen 212 of the fingerprint capture apparatus 100 is depicted. In this figure, eight electrodes are diagrammed that are connected to electrical leads with a single electrode 211e and a single electrical lead 301 being labeled. The platen 212 may have electrodes that are exposed to the environment as depicted in FIG. 2A or electrodes that are covered by a material 231 as depicted in FIG. 2B. As will be detailed later, even if electrodes 211e are not covered by a material 231, electrical leads 301 may be since it may be desirable that such leads are not conductive to the touch of a subject's skin. As described earlier, the electrodes can be made by printing using conductive inks or conductive silicones or patterned using vacuum-deposited material such as ITO or thin metal coatings (e.g., aluminum, gold, or copper). Photolithography may be used to pattern the coatings into the forms of electrodes or electrode leads, but a lower cost approach, scalable to large platen areas, is to use a shadow masking approach as described by S. Franssila Introduction to Microfabrication (John Wiley and Sons, 2004) pp. 229-230.

In shadow masking, a stencil of the pattern that is desired is laid down on the platen surface. The platen is therefore exposed to the coating process in those regions of the stencil that are open and shielded from the coating process in those regions that the stencil blocks the coating process. Alternatively, the electrodes and the electrical leads are patterned onto the platen surface using conductive inks and adhesives with printing processes that include, but are not limited to, silk screening and ink jet printing, such as the services offered by Creative Materials, Inc. (Tyngsboro, Mass.) with their range of conductive adhesives and inks. Still another method of producing the necessary conductive electrodes is to deposit the conductive material in the appropriate electrode and electrical lead pattern into a mold and then to mold the platen material onto the conductive electrodes and electrical leads. For all of these conductive material patterning methods, nonconductive materials can be patterned in conjunction with the conductive materials in order to insulate the electrodes and/or electrical leads as required for the particular electrical design.

Housing 201 of apparatus 100 of FIG. 3A has a top cover member 302 that seals the platen 212 in place. Referring now to FIG. 3B, the underside 311 of the cover member 302 is illustrated. The cover has an opening 310 for the platen, but the electrical leads covering the platen 212 in FIG. 3A extend beyond the cover opening 310 and make contact with leads 313. Leads, such as the one marked as 313 are preferentially fabricated to be sufficiently wide in order to accommodate alignment errors between platen electrical leads 301 and top cover electrical leads 313 during manufacturing. Leads such as 313 are joined to other leads 314 that join to a connector 315, where such circuitry may be part of a flex circuit 312a and 312b that are affixed to the bottom side of cover member 302 which may be a cover to housing 201. The connector 315 is then joined to cable 209d which is the same cable illustrated in FIG. 1.

Referring to FIG. 4A, an exploded view of a fingerprint capture apparatus 100 is illustrated that incorporates a pad 403. As discussed in reference to FIGS. 2A and 2B, the pad, by way of example may be composed of material 215 with integrated electrodes or materials 215 and 231 with integrated electrodes and, as previously discussed, it is preferred that the material touching the skin is an image-enhancing material, such as that which enhances the capture of dry skin. To capture images of dry skin, particularly for an optical imaging system, the pad 403 may, by way of example, incorporate a silicone or urethane material or similar low surface energy material to enable the skin of a finger to make good optical contact with the pad. Alternatively pad 403 may be composed of a compressible material overcoated with a thin non-tacky material (e.g., oxide, semiconductor, metal, or polymer) as described in U.S. Provisional Application No. 61/271,903. Similarly, the pad (or the platen) may have a fine porous material that is impregnated with a second material where the second material is liquid in nature and preferentially a minimum amount of this liquid makes contact with the skin of the finger to improve, rather than degrade the image of the finger, by allowing only the skin ridges to “wet” to the pad surface. For the case of the fingerprint image capture achieved using ultrasonic technology, the pad preferentially allows for good ultrasonic contact of the skin to the pad and may incorporate a gel integrated with a fine porous material. The pad may be in the form of a sticker where a pressure-sensitive adhesive or another form of bonding agent is used to adhere the pad 403 to the platen of the apparatus. Alternately the pad is not mounted to the rest of the apparatus with an adhesive, that although it “wets” to platen 430 and provides either good optical or ultrasonic contact, it may require a pad clamp 402 to hold or retain it in place and/or to ensure good electrical contact with the spoof detection electronics and circuitry contained with the apparatus.

As illustrated in FIG. 4A, the pad 403 is patterned with electrodes 411 that are connected to electrical leads 401. The patterning of the electrodes on the pad can be achieved in a similar manner as the methods described to pattern the platen 212 during the previous discussion relating to FIG. 3A. The pad is placed on surface 430 which for the embodiment depicted in FIG. 4A does not have any electrodes or electrical leads on it. However, in an alternate embodiment, the surface 430 contains electrical leads (as depicted in FIG. 3A) that mate to electrical leads 401 of pad 403 where the pad electrical leads 401 electrically connect the top side of the pad down to the bottom side of the pad, thereby making contact with the surface 430. Similarly, electrodes can be fabricated in the pad 403 that run directly through the pad itself to make contact with electrodes and electrical leads present on the surface 430, thereby eliminating the need for pad electrical leads 401. In another embodiment, the pad is made of an anisotropic conductive material so that there is conductivity substantially in the z axis and minimally along the x or y axes. In this embodiment, electrodes are not required on the pad and can only be on surface 430. For the exemplary electrical geometry illustrated in FIG. 4A, the electrical connection from the pad 403 to the rest of the apparatus 100 is achieved via a pad clamp member 402. This pad clamp may be screwed into the housing 201 (this method is not illustrated), or may be attached via a mechanical hinge that attaches to location 406 of the pad clamp and location 404 of the housing 201. In this manner the pad clamp can be flipped up quickly to allow the interchange of a new pad for a worn or otherwise damaged pad.

FIG. 4B depicts the embodiment wherein a pad clamp member 402 is utilized to hold a pad 403 in place. In this clarifying figure, the pad clamp 402 is attached to housing 201 via a hinge 420 that attaches to location 406 of the pad clamp and location 404 of the housing 201. A spring 422 or similar device may be incorporated into hinge 420 that provides a return force so that although the pad clamp is capable of moving in the direction indicated by the double arrow 421, in its natural state it is biased down towards the pad 403 so that the pad is held in place. Modifications of the depicted embodiment include the addition of locking sliding tabs that go on the opposite side of the pad clamp from hinge 420 that allow the pad clamp to be locked in place, while simultaneously allowing a person to quickly unlock the pad clamp, allowing for the pad to be replaced, but these are considered to be alterations to the present invention that would be obvious to one skilled in the art.

FIG. 4C depicts the bottom side 410 of the pad cover 402 and illustrates how electrical leads 401 make electrical contact with electrical cable 209d of FIG. 1. As depicted in FIG. 4C the bottom side 410 of pad cover 402 contains mating electrical leads 411 that are preferentially sufficiently wide in order to accommodate lateral misalignments of the clamp leads 411 to the electrical leads 401 of the pad resulting from relatives shifts of the pad to pad cover as the pads are replaced.

It is preferable that the apparatus 201 has a mechanism (electrical or otherwise) that can sense when the pad is misaligned relative to the pad cover leads and notifies a user that an adjustment is required. Depicted in FIGS. 4A and 4C are the sufficient electrical lead arrangements to determine if there is such a misalignment in the y direction. In FIG. 4A, a set of horseshoe shaped electrical leads 408 are present on pad 403. These leads 408 make contact with leads 420 and 421 of the underside of the pad cover diagrammed in FIG. 4C. By checking the electrical conductivity between leads 420 and 421, the fingerprint capture apparatus can determine if the pad 403 is misaligned in y. Similar electrical lead arrangements may be provided to determine misalignments in the x direction as well if required.

Leads such as 411 are connected to secondary leads 412 that may be thinner, which connect to electrodes 414 that connect to electrical contact area 405 diagrammed in an isometric view in FIG. 4A and in a side view in FIG. 4B. Electrodes 414 are preferentially sufficiently large to account for any misalignments relative to the housing cover that may occur and potentially break the electrical connection between electrodes 414 (which there are an exemplary six number of electrical pads illustrated on each side of the clamp bottom) and those of electrical contact area 405 (with an exemplary six electrical pads matching the position and size of those of the clamp bottom). Contact area 405 is connected to electrical cable 209d which is connected to electrical spoof circuit board 207c and electrical processor 210b as described in context of FIG. 1.

FIG. 5 illustrates an arrangement of electrodes 211 for the capture of multiple fingers. The rectangular border marked as 501 may represent the border of a pad or of the apparatus platen 212. As illustrated, electrodes 211 are arranged so as to analyze the fingerprints of four separate fingers with said fingers being depicted as the dashed outlines 505a, 505b, 505c, and 505d. The electrodes are grouped within fingertip rectangular regions 504a, 504b, 504c, and 504d wherein each fingertip rectangular region electrically probes a single finger tip. Within each fingertip rectangular region, electrodes, such as 502a, 502b, and 502c are contained. Although only six electrodes of equal shapes and areas are depicted per fingertip rectangular region any number may be utilized and the electrodes may include shapes such as rectangles, lines, and circles and all electrodes need not be of the same shape or size. Electrodes 502a, 502b, and 503c are connected to electrical leads 503a, 503b, and 503c, respectively and are depicted as running towards the edge of boundary 501 so as to be connected to cable 209d of FIG. 1. These electrical leads may alternatively run straight down in the z direction rather than run to the boundary 501 edges to connect to cable 209d. The fingers as outlined may be in physical contact with electrodes so that DC resistivity can be measured, or may be a certain height z₀ above the electrodes such as the case for an AC impedance sensor. In the case where the apparatus operates with the fingers not in direct contact with the electrodes, the subject may suspend his or her fingers over the platen as in FIG. 2C or may be in contact with the platen, but not in direct contact with the electrodes if the electrodes are coated with a dielectric layer as depicted in FIG. 2B.

With the electrode geometry of FIG. 5, each of the subject's fingertips are preferably placed over each of the fingertip rectangular regions. To aid in this alignment, the fingertip rectangular regions (504a-d) are preferentially visible to the subject. By way of example, the regions may be colored differently from the rest of the platen or lights under the platen illuminate these regions such that they are visible to the subject. Also, since in order to make the apparatus symmetric for left and right hands of an individual, it is preferable that the fingertip rectangular regions are symmetry in x. In other words, it is preferred that rectangular regions 504a and 504d are located at the same y coordinate and similarly that rectangular regions 504b and 504c are located at the same y coordinate. By arranging fingertip rectangular regions in this manner these regions can accommodate a wide range of hand and finger sizes by having the subject rotate their right hand to the left when placing it over the electrodes and by rotating their left hand to the right when placing it over the electrodes. By rotating the hand, this addresses the issue of the pinky finger being in general significantly shorter than the index finger, thereby accommodating both left and right hands with the same electrode arrangement. Mechanical dividers at the fingertip side of 501 may be incorporated into the apparatus 100 in order to guide a subject's hands. Although not shown in FIG. 5, these mechanical dividers have been implemented in current fingerprint devices that use a separate camera to image the left two fingers of a hand versus the right two fingers of a hand, as in the case of four-finger fingerprint scanner devices such as the TouchPrint™ 4100 (L-1 Identity Solutions, Stamford, Conn.) and ID 500™ (Cross Match Technologies, Inc., Palm Beach Gardens, Fla.). For the implementation of such mechanical dividers, care should be taken to ensure that the dividers still allow for sufficiently rotation of the left and right hand to allow the correct fingers to be positioned over the correct rectangular region. Although electrodes may be patterned down to 1 micron and smaller size through the aid of photolithography, it is preferable to integrate electrodes into a fingerprint capture device using low-cost fabrication processes. If cost is not an object, the area 501 can be filled with electrodes and provided they are small enough and the fabrication process has sufficient resolution, that leads can be patterned in the same area. Since an image of the fingerprint is being captured by the apparatus, the apparatus's processor(s) may determine whether or not the fingers are properly positioned on rectangular fingertip areas, thereby avoiding the scenario of reporting a finger spoof because of a fingertip missing or only partially covering the required fingertip area.

Preferably the electrodes, such as 502a, 502b, and 502c allow for impedance measurements of the finger at different electrode spacings. This has the advantage that the impedance of the dermal tissue of the finger can be detected which changes least from person to person or within the same person versus the epidermal portion of the skin which, being the outermost layer of skin, can change impedance characteristics due to how dry, sweaty, or calloused this layer of skin is at the time of the electrical observation.

Another arrangement of electrodes 211 is shown in FIG. 6A. The advantage of the electrode arrangement of FIG. 6A is that the fingers (represented by dashed outlines 505a and 505b) do not have be aligned to specific electrodes as in FIG. 5. The second advantage of the arrangement of FIG. 6A is that there are a reduced number of electrical leads 603a due to the inclusion of long rectangular electrodes 604a in addition to the small rectangular electrodes 602a. By measuring the impedance value across electrodes 602b to 604a and from 602d to 604a, the impedance of two separate fingers may be independently determined. Likewise, for a given finger, the impedance across electrode pairs such as 602b to 604a and 604a to 602a may be determined in order to measure across different distances of the subject's fingers in order to better match the measured impedance values to those of a human. In this manner, long rectangular electrodes, such as shown by 604a and 604b, are oriented on the platen to receive signals from multiple ones of fingers when present on the platen. The electrode pattern shown in FIG. 6A may be extended along the platen for detecting electrical signals of more than the two fingers illustrated (e.g., extended such that four fingers can be measured).

For the case of the electrodes in FIG. 6A being used in direct contact with a finger, the electrode such as 602e needs to be exposed to the outer surface of the apparatus, while the electrical leads 603b need to be insulated. The area A of FIG. 6A is represented in FIG. 6B as a top-down view and in FIG. 6C as a cross-sectional view. The electrode 602e is patterned on surface 501, but then preferentially overcoated with an insulating layer 615. In this manner, although portions of electrode 602e inside of boundary 612 can be touched by a finger, portions of the electrode or electrical lead outside of boundary 612 are insulated. For example, electrode 610 and electrical lead 603 may be fabricated out of vacuum-deposited ITO and patterned using a shadow masking process [see, for example S. Franssila Introduction to Microfabrication (John Wiley and Sons, 2004) pp. 229-230] and the insulating layer can be sputtered SiOₓ that can be registered and patterned relative to ITO electrodes and leads using a shadow masking process. Although electrical lead 603b is represented by a solid line and electrode 610 is represented by hash marks, they may be fabricated from the same conductive material. Thus insulating material is provided over at least the leads and provides openings for the electrodes (preferably overcoated as shown), and a subject's fingers can contact the electrodes via the openings.

Alternatively, the electrodes may be overcoated and AC impedance properties such as capacitance of the skin topology are measured with the electrodes. Schematics of two exemplary cross-sections of electrodes operating in the mode are depicted in FIGS. 6D and 6E. In FIG. 6D, rather than patterning the insulating layer, the entire platen is coated with a material 620 (covering both electrodes and their leads). The material 620 as previously described may be an insulating dielectric material such as SiOₓ that can be vacuum or flame deposited. Alternatively or in addition to an image enhancing material 621 may be deposited upon the entire platen surface. As described earlier, for an optical system, the material may by way of example be a silicone or urethane material that improves the optical contact of dry skin to the platen surface. In order to sense electrical properties of skin directly above the electrodes, it is preferable that the electrodes such as 602e are significantly larger in area than the electrical traces or leads 603b so that primarily the impedance of the skin above the electrodes are measured, rather than of the skin directly above the traces.

To increase the discrimination of the impedance measurement above the electrode versus that above the electrical leads, the electrodes as illustrated in FIG. 6E may be used. Referring to FIG. 6E, the electrodes (e.g., 602e) and electrical leads (e.g., 603b) are patterned and then a material 622 (preferably insulating) is patterned such that the platen surface except for the electrodes (or alternatively, just the electrical leads) are overcoated. A second material 623 for image enhancement is then deposited where the image enhancing material may or may not be the same material as material 622. The result of the architecture of FIG. 6E is that the electrical leads 603e are separated further from the skin topology that is pressed against the platen compared to the electrodes 602e to decrease the feedback of the electrical leads during any impedance or voltage signal measurement of the skin topology. Thus, unlike FIG. 6D, the insulating material is substantially thicker over the leads than the electrodes.

Referring to FIG. 6F, an alternative to FIG. 6E is illustrated for decreasing the sensitivity of the electrode 602e versus its lead 603b. First a material 640 is patterned on platen surface 212 where the purpose of the material is to raise the electrode 602e height versus that of the electrical lead 603b that are subsequently patterned. Next a material 641 is optionally patterned such that electrode 602e is still exposed. Alternatively or in addition to, a material 642 is coated across the entire platen. Material 642 (same as material 623 of FIG. 6E) may be a planarizing coating that is spray, dip, or meniscus applied to the surface and may be an image-enhancing material which for the case of an optical fingerprint scanner may by way of example be urethane or silicone. Alternatively, conductive vias (represented by dashed lines 652 through material such as 640 may be created such that the electrode sits on top of the conductive via. In this embodiment, the portion of the electrical lead 651 is not required since it is electrically connected to the electrode 602e through via 652. Thus, the electrodes are disposed upon an insulating material, and their leads downwardly extend from the electrodes toward said platen, where the insulating material covers at least the leads and has openings for said electrodes (preferably overcoated). Preferably in all of example of FIGS. 6C-6F, a material for enhancing images captured by the imaging system, as described earlier, may be applied as a top coat or layer over leads and electrodes extending there from, and/or the insulating material also provides an image enhancing material.

It should be understood that in FIGS. 6C-6F that although materials 615, 620, 621, 622, 623, 640, 641, and 642 are drawn as single-layer materials in FIGS. 6C-E, additional layers of materials may be present in order to promote adhesion or reduce the tackiness of an optional top layer to increase dry finger capture. Additionally, electrodes 602e and leads 603b may be composed of one or more materials depending upon such requirements as adhesion, conductivity, or use and storage environment.

For the non-direct contact electrode of FIGS. 6D-6F, in the analysis of the liveness signals by processors 210b, the thickness(es) and electrical property(ies) of the material(s) above the electrodes should be taken into account since the thicker and more electrically insulating the materials above the electrodes are the lower the signals detected will be. In particular, it is critical that the tolerance on the thickness values of the layers do not exceed an anticipated thickness of a spoof membrane (e.g., a fake fingerprint) that is placed on a subject's real fingerprint or create such a large change in impedance measurement of a skin that a spoof may be incorrectly identified. If the manufacturing thickness tolerances are significant, than it is critical that the overcoated electrodes are calibrated during factory assembly so that the liveness electronics and processors 210b can record factory settings that indicate a live finger versus a spoof and the settings may vary from fingerprint scanner to fingerprint scanner due to the variations in manufactured coating. For the case of an image-enhancing pad to be field-replaceable, if the thicknesses variations of the optional material covering the electrodes of the pad vary significantly a field calibration process may be required wherein a control object is placed on the pad after replacement, electrical measurements taken, and then the electrical measurements provide the basis for the threshold value decision for determining a spoof of live finger upon subsequent subject acquisition events.

In order to increase the surface area of surface 501 that is available for electrodes without increasing the resolution of the patterning process required, multiple layers of conductive and non-conductive materials may be used as shown in FIG. 7A. The electrical properties of a finger 505a can be checked by making impedance measurements across electrodes 701a and 604b and across electrodes 701b and 604b. The different distances between each of these respective electrodes allows impedance measurements to be taken across varying distances of skin, thereby allowing for the impedance value of the dermal versus the epidermal skin to be distinguished. Electrodes 701a and 701b are connected to electrical leads 705a and 603a which although shaded differently, function identically by making electrical contact between the electrodes and the rest of the apparatus. The shading difference in the figure between the two electrical leads is meant to distinguished the two types of electrical leads and is clarified by examining area B of FIG. 7A which is illustrated in a cross-section in FIG. 7B.

FIG. 7B illustrates two levels of electrodes in the z-direction, and such may be extended to numbers of electrode levels higher than two. As illustrated, electrode 701c and electrical lead 705b are patterned on platen surface 212. Then an insulating layer 615b is patterned such that at least a portion of 701c is exposed. Afterwards, electrode 701d and electrical lead 603c is patterned on the resultant film stack and then an insulating layer 615a is patterned such that it covers and thereby insulates the electrical line 603c but leaves at least some of electrode 701d uncovered. The electrode stack illustrated in FIG. 7B is for an apparatus that requires the finger to be in direct contact with the electrodes, however, the electrode arrangement and the patterning process need not be restricted to such technology. Alternatively the insulating layers 615a and 615b can completely cover both of the electrical leads and the electrodes in order to serve as an electrical surface for measuring the capacitance of an object approaching platen 212. Alternatively, the patterning processes and electrode described in connection with FIGS. 6D-6F may be incorporated into the electrode depicted in FIG. 7B. The height along the z-axis of these layers illustrated in FIG. 7B is negligible to the user placing his finger on the surface 501 since each coating layer need only be a fraction of a micron to provide the necessary conductivity or insulating properties. As discussed previously, the non-conductive material may be chosen such that the absorption (optical or ultrasound) matches approximately that of the electrically conductive material in order to create an image that is more uniform across the image capture area of the apparatus.

FIG. 8A depicts the overlay of a bright-field optical image of a fingerprint 800 relative to the electrodes of the apparatus that are sensing the finger's electrical characteristics. As previously noted, it is preferential that multiple electrodes, for example 801a, 801b, and 802 probe the finger so that impedance measurements can be made across multiple values of electrode separation. In FIG. 8B, a close-up view of the fingerprint image overlaid on an electrode position is illustrated. The particular electrodes the fingerprint is over, the electrodes the fingerprint is not over, and which ones are partially covered, may be determined by processor(s) 210a and/or computer system 102 analyzing the grayscale along regions of pixels in a captured image associated with each electrode location as stored in memory accessible to processor(s) 210a and/or computer system 102. As indicated in FIG. 8B, the fingerprint image, partially covers the electrode having outline 801a. This may be the case of an electrode requiring physical contact with the finger or for an electrode that is overcoated with an insulator later (for example a capacitance touch screen) or for an electrode that allows the finger to be a certain distance away from the electrode in air, as described earlier in connection with FIGS. 2A-C.

For electrode 801a in FIG. 8B the area of the captured image should be checked for electrodes to the right of fingerprint boundary 805 and contained within the electrode boundary 801a. Since a fingerprint is a 3D topology and contains ridges and valleys, it is less accurate to ignore the contact area of the ridges, since depending upon skin pressure and moisture level on the skin, the ridges may be narrower or wider across different people and even the same person. The image should be segmented to locate fingerprint boundaries about the finger tips and the electrodes are used for spoof detection that are located in the area of the ridges that make contact with the apparatus. For the bright-field optical fingerprint image of FIG. 8B, the areas where the fingerprint makes contact with the electrodes are the dark pixels pointed to by tag 804, while the bright pixels, such as those indicated by 803, are the valley areas. Such a fingerprint image can be captured by the fingerprint capture apparatus 100 as illustrated in FIG. 1. Alternately, the illumination source 203 of apparatus 100 of FIG. 1 can be reconfigured to produce dark-field images, such as described for example in U.S. Pat. No. 5,233,404, and then the histogram of the fingerprint image of FIGS. 8A and 8B would be inverted and the calculation of the area of contact with electrodes would be through the counting of bright pixels instead as these would indicate instead where the fingerprint ridge is making contact with the platen surface and therefore the electrodes (or regions above the electrodes in the case of an overcoating layer).

Referring to FIG. 9A, another electrode is shown which is denoted an electrode 900, such as might be used in place of electrode 602e from FIG. 6A. Unlike electrode 602e depicted in FIG. 6B, electrode 900 consists of a mixture of conductive areas 903 and non-conductive areas 904. Although non-conductive areas 904 are drawn as rectangular areas arranged in a rectangular grid, other arrangements may be used, such as circular non-conductive areas arranged in a hexagonal array pattern. Shaping the outer boundary of electrode 900 and non-conductive areas 904 as circles or ellipses or other shapes that do not have sharp corners may be advantageous in terms of reducing unwanted electrical effects at higher AC frequencies. The advantage of having electrodes composed of micropatterned nonconductive areas is so that conductive areas do not compromise the imaging system's ability to capture an image. In this scenario, having a reduced distance between non-conductive areas captured by the imaging device of the apparatus allows the image processing software and/or electronics of the apparatus to better interpolate between areas of the image that were imaged through non-conductive areas in order to improve the image quality of the captured fingerprint. For example, if the electrode patterning (or alternatively or in addition to, individual electrodes themselves) have a size that is smaller than the resolution of the system, then it may not matter if the conductive material does not transmit optical or ultrasonic waves at the desired level. To further clarify, for the case of a 500 ppi (points per inch) resolution (i.e., each pixel is 50.8 μm×50.8 μm) optical fingerprint scanner, if the conductive areas of the electrode are only 10 μm wide with 10 μm non-conductive holes (or an individual electrode is only 10 μM wide) the optical system will not see the electrode even if it is opaque. Instead it will see a pixel intensity value that is the average of the light from the electrode areas and the non-electrode areas. The advantage of this approach, though at the cost of requiring a finer resolution patterning processes, is that the electrodes can be composed of materials that are opaque to the scanning technology used (i.e., metals such as Al or Ag can be used despite these metals not transmitting light at visible or near infrared wavelengths). Similar alternating of conductive and non-conductive areas can be performed with the electrical leads as well as indicated by conductive areas 902a, 902b, and 902b, and non-conductive areas 901a and 901b. The patterning of the electrodes and electrical leads may be achieved using shadow masking as previously described, but may also be achieved through photolithographic processing steps. In order to ensure that the illumination emanating from the platen is uniform, “dummy” electrodes or leads may be patterned on the surface which are not connected to the liveness detection board 207c or even to the edge of the platen.

Illustrated in FIG. 9B is an electrode layout where the features of the electrodes and the leads are smaller than the resolution of the imaging system (which by way of example may be optical or acoustic). Due to the large number of electrodes present in a given area to achieve this, not all of the electrodes need to be read in order to determine liveness of the subject's skin. As depicted in FIG. 9B, half of the electrodes (e.g., 920) are not connected and half of the electrodes (e.g., 921) are connected to leads 922. Alternatively all of the electrodes 920 and 921 maybe connected to electrical leads such as 922, but all of these leads are not connected to connector 209d or if they are they may not be all read by liveness circuitry 210b. In order for the imaging system to see a relatively uniform illumination or response from the entire platen 923, the electrodes 920 and 921 along with leads 922 fill the platen area with approximately equal density.

Illustrated in FIG. 9C is another embodiment of an electrode layout where the features of the electrodes and the leads are smaller than the resolution of the imaging system. In this embodiment, platen 930 is filled with electrodes 934 that are connected to electrical leads 935 and electrodes 936 that are not connected to electrical leads (or are, but are not necessarily read out to determine liveness). Electrodes 934, as also illustrated in FIG. 9A, are composed of conductive areas 932 and non-conductive areas 933 that may have features beneath the resolution of the imaging system of apparatus 100. In this manner, the imaging system see a uniform platen response and therefore requires less field flattening, compared to a geometry where the electrodes and non-electrode patterned areas are larger than the limiting resolution of the imaging system. In FIG. 9C, electrodes may have different sizes (e.g., 934 versus 931) to aid in the liveness discrimination. Other pattern geometry than those of the electrodes and leads shown in the FIG. 9B or 9C may be used. Combination of the electrodes shown in FIG. 9B and FIGS. 9A and 9C may be used in or on the platen, or a pad upon the platen. Although electrodes are shown in figures as pads with leads, electrodes of other shapes may be use to acquire signals for liveliness detection.

FIG. 10 depicts an isometric view of fingerprint capture apparatus 100 which enables measure of electrical signals through the subject's heart by processor(s) 210b using finger(s) of two separate hands of a subject. The apparatus's platen 212 has two platen areas 212a and 212b that are patterned with electrodes or accepts a pad containing electrodes as previously discussed, but where electrical signals in addition to being measured across electrodes contained within the same platen are also measured across electrodes from the two different platen areas 212a and 212b. Each platen area 212a and 212b accepts the finger(s) of the subject and is separated by divider 1001 from the other where divider 1001 is sufficiently tall and/or long so as to discourage a subject from laying fingers of a single hand on both platen areas 212a and 212b. Platen areas 212a and 212b may be physically distinct platens, as drawn, or they may be portions of the same platen 212 that are merely divided on top by divider 1001. The divider is provided primarily as a deterrent for the subject to place fingers of one hand on both platens since if the subject does, the electronics (e.g., processor 210b) will sense a different impedance due to the significant difference in path length of the electrical signals going from one finger to a second finger of the same hand versus electrical signals going from one finger of one hand to a second finger of the opposite hand. Instead of two platen areas 212a and 212b, or two separate ones of platen 212 providing such areas on the same apparatus 100, two separated ones of apparatus 100 may be connected to the same computer 124 which measures electrical signals from electrodes of the first one of the apparatuses 100 to electrodes of the second one of the apparatus 100, where the subject touches each of the two apparatuses with a different hand (or finger(s) thereof) to detect the subject's heart pulse. The subject's heart pulse detected represents a liveness parameter which either processors 210b or computer system 124 may be programmed to determine subject's liveness by one or more of being present or in range of a typical living human.

From the foregoing description, it will be apparent that there have been provided an improved apparatus and method for the use of contact as well as non-contact electrodes that are integrated into a fingerprint scanner, i.e. an apparatus capturing the image of a fingerprint (or similar 2D or 3D skin topology) where the electrodes are incorporated for the purposes of liveness analysis of the skin presented to the apparatus. Such fingerprint scanner with the improvement provided by the present invention may be for be used for biometric identification, verification, and/or identification of a subject. Variations and modifications in the herein described apparatus, method, and system in accordance with the invention will undoubtedly suggest themselves to those skilled in the art.

1. An apparatus for identification of fake fingerprints in a fingerprint scanner having an imaging system capable of capturing an image of multiple fingerprints via a platen, said apparatus comprising:
a plurality of electrodes disposed in or upon the platen; and
means for detecting liveness of fingers when presented to the platen in accordance with electrical signals provided from said electrodes.

2. The apparatus according to claim 1 wherein said plurality of electrodes are configured into different groups of said electrodes, and at least part of the fingers when presented to said platen are spatially oriented with respect to different ones of said groups of said electrodes.

3. The apparatus according to claim 2 wherein said plurality of electrodes are spatially oriented in different ones of said groups of said electrodes on said platen with only the tips of the fingers when presented to said platen.

4. The apparatus according to claim 1 wherein at least one of said electrodes is oriented on said platen to receive signals from multiple ones of said fingers when presented to said platen.

5. The apparatus according to claim 1 wherein said plurality of electrodes disposed in or upon said platen are in non-contact with the fingers when presented to said platen by one of a gap between said electrodes and the fingers, or at least a material for enhancement of the image of the fingerprints when captured.

6. The apparatus according to claim 1 wherein said electrodes each have a lead extending from the electrode, and said apparatus further comprises a member adjacent to said platen having at least one lead extending the lead from each electrode to communicate signals from said electrodes to said detecting means.

7. The apparatus according to claim 1 further comprising a pad upon said platen having said electrodes and the fingers are presented to said platen via contact with said pad.

8. The apparatus according to claim 7 wherein said pad is removable and replaceable with a different pad upon said platen.

9. The apparatus according to claim 7 wherein said electrodes each have a lead extending from the electrode, and said apparatus further comprises:
a clamp member adjacent said platen having at least a lead extending to the lead of each electrode to communicate signals from said electrodes to said detecting means in which said clamp member retains said pad in place.

10. The apparatus according to claim 7 wherein said electrodes each have a lead extending vertically downward in said pad to provide said signals to said detecting means.

11. The apparatus according to claim 7 where said pad has anisotropic material and signals travel vertically via the anisotropic material to said electrodes on or in the platen in response to fingers placed onto the pad.

12. The apparatus according to claim 7 wherein said pad has a first porous material providing one of second liquid or gel material in which only ridges of fingers are wet by said second material when the fingers are in contact with said pad.

13. The apparatus according to claim 7 wherein said pad comprises a layer of material over at least said electrodes for enhancing imaging of fingerprints captured by said imaging system via said platen.

14. The apparatus according to claim 7 wherein said pad has at least one layer of non-tacky material over said electrodes, said non-tacky material being optically transparent for imaging there through by said imaging system.

15. The apparatus according to claim 14 wherein said non-tacky material enhances imaging of fingerprints when captured by said imaging system via said platen.

16. The apparatus according to claim 1 wherein said platen has a first porous material providing one of second liquid or gel material in which only ridges of fingers are wet by said second material when the fingers are in contact with said pad.

17. The apparatus according to claim 1 wherein said electrodes have leads extending from each electrode, and said signals are provided via at least the leads to said detecting means, and said apparatus further comprising an insulating material over at least the leads.

18. The apparatus according to claim 17 wherein said insulating material has openings for at least part of said electrodes.

19. The apparatus according to claim 18 wherein said fingers contact said electrodes via said opening when said fingers are presented to said platen.

20. The apparatus according to claim 18 further comprising material over both said insulating material and said openings for enhancing images captured by said imaging system via said platen.

21. The apparatus according to claim 17 wherein said insulating material covers both said leads and said electrodes to said leads.

22. The apparatus according to claim 21 further comprising material over said insulating material for enhancing images captured by said imaging system via said platen.

23. The apparatus according to claim 21 wherein said insulating material enhances images captured by said imaging system via said platen.

24. The apparatus according to claim 21 wherein said insulating material is substantially thicker over said leads than said electrodes.

25. The apparatus according to claim 17 wherein said electrodes are disposed upon said insulating material, and said leads downwardly extend from said electrodes toward said platen, said insulating material covers at least said leads and has openings for at least part of said electrodes.

26. The apparatus according to claim 25 further comprising material over said insulating material and said openings for enhancing images captured by said imaging system via said platen.

27. The apparatus according to claim 1 wherein said electrodes have leads extending from each electrode, and said signals are provided via at least the leads to said detecting means, and said imaging system has a resolution for imaging, and said one or more of said electrodes and said leads are of a size smaller than the resolution of said imaging system.

28. The apparatus according to claim 1 wherein one or more of said electrodes are disposed at different heights in or upon said platen.

29. The apparatus according to claim 1 further wherein the fingers contact said platen and one or more of said electrodes when presented to said platen.

30. The apparatus according to claim 1 further comprising said imaging system operative for providing an optical, ultrasonic, or capacitive image of fingerprints when presented via said platen.

31. The apparatus according to claim 30 wherein said detecting means utilizes signals only from one or more different ones of said electrodes in said image where fingerprints are present for determining liveness of the fingers.

32. The apparatus according to claim 1 wherein said means further comprises:
means for determining one or more liveness parameters in accordance with said signals from electrodes in accordance with said one or more liveness parameters being present or not.

33. The apparatus according to claim 1 wherein said means further comprises:
means for determining one or more liveness parameters in accordance with said signals from electrodes in accordance with said one or more liveness parameters being in or outside a range of a living subject.

34. The apparatus according to claim 1 wherein said means further comprises:
means for determining one or more liveness parameters in accordance with said signals from electrodes in accordance with said one or more liveness parameters being in or outside a range for the subject.

35. The apparatus according to claim 1 wherein said electrical signals provided from said electrodes are in accordance with one or more of real skin impedance, imaginary impedance, skin temperature of the subject, heart pulse of the subject, skin resistivity, capacitance, or induction.

36. The apparatus according to claim 1 wherein said electrical signals provided from said electrodes are in accordance with at least complex impedance values calculated as a function of electrical frequency.

37. The apparatus according to claim 1 wherein said electrodes are transparent to the imaging system.

38. The apparatus according to claim 1 wherein at least one of said electrodes detect a heartbeat of a subject when skin of the subject is presented to said platen.

39. The apparatus according to claim 1 wherein at least one of said electrodes sense the ambient temperature and the temperature of skin when touching the platen.

40. The apparatus according to claim 1 wherein said electrodes comprise a phased array for at least one of transmitting or receiving electrical signals from a specific location or region of the fingers when presented to said platen.

41. The apparatus according to claim 1 wherein said platen has a first platen area and a second platen area, each having different ones of said electrodes, and said signals are received said are representative of a heart pulse of the subject when one of more fingers of a first of hand of the subject are presented to said first platen area, and one of more fingers a second hand of the subject are presented to said second platen area.

42. The apparatus according to claim 41 wherein said first platen area and said second platen are along different ones of said platen.

43. The apparatus according to claim 1 further comprising a conductive bar which makes contact with the fingers away from the fingertip when fingers are presented to said platen, and said bar comprises means for detecting temperature and sending a signal representative of detected temperature for use by said detecting means to check whether the detected temperature is in range typical of a living subject.

44. The apparatus according to claim 1 further comprising means for sensing ambient temperature and providing said sensed ambient temperature to said detecting means when need to compare with temperature sensed by fingers of the subject.

45. An apparatus for fingerprint image acquisition and liveness detection comprising:
a platen;
an imaging system operative for providing an optical, ultrasonic, or capacitive image of the one or more fingerprints via said platen;
electronics for detecting electrical signals from skin in which the one or more fingers imaged are one of non-contact with said electronics or contacts said electronics via material which enhancing imaging of the one or more fingerprints; and
one or more processors to analyze the received electrical signals to determine liveness of the skin when presented to the apparatus.

46. The apparatus according to claim 45 wherein said electronics comprise electrodes disposed in or on said platen.

47. The apparatus according to claim 46 wherein said platen comprises a plurality of layers and said electrodes are below at least the uppermost layer, and said uppermost layer which contacts skin when presented to said apparatus for imaging by said imaging system is of material which enhancing imaging by said imaging system.

48. The apparatus according to claim 46 wherein said electrode are arranged upon said platen to receive signals from more than one finger when presented in said platen.

49. The apparatus according to claim 46 wherein said electrodes sense the complex impedance of one or more of dermal or epidermal skin when touching said platen by sending electrical signals through the electrodes.

50. The apparatus according to claim 46 wherein said electrodes sense the ambient temperature and the temperature of the skin when touching the platen.

51. The apparatus according to claim 46 wherein said plurality of electrodes are configured into different groups of said electrodes, and when multiple fingers are presented to said platen, said fingers are spatially oriented with respect to different ones of said groups of said electrodes.

52. The apparatus according to claim 46 wherein at least one of said electrodes is oriented on said platen to receive signals from multiple ones of said fingers when presented to said platen.

53. The apparatus according to claim 46 wherein said one or more processors utilize signals only from ones of said electrodes disposed with respect to locations of said fingerprints in said image are used for determining liveness of the skin.

54. The apparatus according to claim 46 wherein said platen comprises a plurality of layers at least of said layers being of non-tacky material over said electrodes, said non-tacky material being optically transparent for imaging there through by said imaging system.

55. The apparatus according to claim 46 further comprising providing a pad upon said platen having said electrodes and the fingers are presented to said platen via contact with said pad.

56. The apparatus according to claim 45 wherein said optical imaging system captures an image of one or more fingerprints non-touching said platen when presented to said platen, and said electronics comprise a phased array for at least one of transmitting or receiving electrical signals from a specific location or region of the skin or finger presented to said platen.

57. The apparatus according to claim 45 wherein said optical imaging system captures an image of one or more fingerprints touching said platen when presented to said platen.

58. The apparatus according to claim 45 wherein said electronics detect a heartbeat of a subject when skin of the subject is presented to said platen.

59. The apparatus according to claim 45 wherein said one or more processors to analyze the received electrical signals to determine one or more liveness parameters in accordance with one or more of said liveness parameters being present or not, being in or outside a range of a living subject, or being in or outside a range for the subject.

60. A method for imaging fingerprints comprising the steps of:
presenting a plurality of fingers to a platen having a plurality of electrodes in or upon said platen;
capturing an image of fingerprints of the fingers presented; and
detecting liveness of said fingers in accordance with electrical signals provided from said electrodes.

61. The method according to claim 60 further comprising providing a pad upon said platen having said electrodes and the fingers are presented to said platen via contact with said pad.

62. The method according to claim 60 wherein said detecting step is carried out in accordance with signals representing one or more of real skin impedance, imaginary impedance, skin temperature of the subject, heart pulse of the subject, skin resistivity, capacitance, or induction.

63. The method according to claim 60 providing a layer over said platen and electrodes of material which enhances said image of a fingerprint from each finger by said capturing step.

64. A method for imaging of at least one fingerprint comprising the steps of:
presenting at least one finger to a platen having a plurality of electrodes in or upon said platen in which said finger is non-contact with said platen;
capturing an image of fingerprint of the finger presented; and
detecting liveness of said finger in accordance with electrical signals provided from said electrodes.
